(19) United States
(12) Patent Application Publication
(10) Pub. No.: US 2009/0281061 A1
(43) Pub. Date: Nov. 12, 2009

(54) Impaired Alleles of Genes Involved in Metabolic Pathways and Methods for Detecting and Using the Same

(75) Inventors: Nicholas Marini, Greenbrae, CA (US); Jasper Rine, Point Richmond, CA (US)

Correspondence Address: King Spalding LLP, 4 Embarcadero Center, Suite 3500, San Francisco, CA, 94111, US

(73) Assignee: The Regents of the University of California, Oakland, CA (US)

(21) Appl. No.: 12/413,506

(22) Filed: Mar. 27, 2009

Related U.S. Application Data
(60) Provisional application No. 61/040,020, filed on Mar. 27, 2008.

(51) Int. Cl. C12Q 1/68; A61K 31/675; C07H 21/04; C40B 40/08; A61P 35/00; A61P 25/00; A61P 9/00

(52) U.S. Cl. 514/89; 435/6; 536/23.1; 506/17

(57) ABSTRACT

The invention is directed to enzyme variants, responsiveness thereof to cofactors, and in vivo assays for testing the activity of enzyme variants as well as the responsiveness thereof to cofactors.

### CROSS REFERENCE TO RELATED APPLICATIONS

This application claims the benefit of provisional application U.S. Ser. No. 61/040,020, filed Mar. 27, 2008, which is incorporated herein by reference in its entirety.

### GOVERNMENT FUNDING

This invention was made with government support from the Defense Advanced Research Projects Agency and the U.S. Army Research Office (#W911 NF-06-1-0166) and the National Institutes for Health (GM072859). The United States government does have certain rights in the invention.

### FIELD OF THE INVENTION

The invention concerns enzyme variants impacting metabolism, functional sensitivity thereof to cofactors, and assays for detecting impaired alleles encoding such enzyme variants and determining the sensitivity thereof to cofactors.

### BACKGROUND

The folate/homocysteine metabolic pathway constitutes a network of enzymes and enzymatic pathways that metabolize folate and/or affect homocysteine. The pathways are linked via the methionine synthase reaction, and marginal folate deficiencies in cell cultures, animal model systems and in humans impair homocysteine remethylation (see, for example, Stover P J. 2004. Physiology of folate and vitamin B12 in health and disease. Nutr Rev 62:S3-12).

Folate inadequacy has been linked to neural tube defects (“NTDs”) as well as other birth defects and adverse pregnancy outcomes, such as orofacial clefts, pre-eclampsia, pre-term delivery/low birthweight, and recurrent early spontaneous abortion (see, for example, Mills et al, 1995. Homocysteine metabolism in pregnancies complicated by neural tube defects. Lancet 345:149-1151). Folate inadequacy has also been associated with cardiovascular disease, coronary artery disease, ischemic stroke, atherosclerosis, thrombosis, retinal artery occlusion, Down's Syndrome, colorectal cancer, breast cancer, lung cancer, prostate cancer, depression, schizophrenia, Alzheimer's Disease/Dementia, age-related macular degeneration, and glaucoma.

All the metabolic steps in the folate/homocysteine metabolic pathway are potentially relevant to conditions and diseases associated with folate inadequacy and/or homocysteine metabolism. Enzymes involved in folate/homocysteine metabolism that are implicated include, e.g., bifunctional enzyme AICAR Transformylase and IMP Cyclohydrolase (ATIC), glycinamide ribonucleotide transformylase (GART), methionine adenosyltransferase 1, alpha (MATlA), methionine adenosyltransferase 11, alpha (MAT2A), methylenetetrahydrofolate reductase (MTHFR), and methenyltetrahydrofolate synthetase (MTHFS). Folate inadequacy also impairs methylation mediated by S-adenosyl-methionine (“SAM”), which is an allosteric inhibitor of both MTHFR and CBS (see, for example, Kraus et al., 1999. Cystathionine β-synthase mutations in homocystinuria. Hum Mut 13:362-375; Daubner et al., 1982. In Flavins and Flavoproteins, eds. Massey, V. & Williams, C. H. (Elsevier, N.Y.), pp. 165-172). Elevations in the S-adenosyl-homocysteine:S-adenosyl-methionine (SAH/SAM) ratios have been proposed in the mechanism of NTD development.

5,10-Methylenetetrahydrofolate reductase (MTHFR) is involved in the folate-dependent multistep pathway in which homocysteine is converted to methionine. Decreased conversion of homocysteine can lead to hyperhomocysteinemia.

Several rare mutations of MTHFR have been identified that are associated with clinical MTHFR deficiency, an autosomal recessive disorder. The clinical symptoms of MTHFR deficiency are highly variable and include developmental delay, motor and gait abnormalities, seizures, and premature vascular disease.

Common polymorphisms of MTHFR have also been described, including the functionally impaired allele A222V. The genetic association of common polymorphisms with disease has not been consistent. This may be due in part to compensatory effects of folate availability that mask an underlying risk of disease, as well as the contribution of as yet unidentified low frequency impaired alleles to such diseases. Interestingly, common polymorphisms have been associated with individual variation in the efficacy and toxicity of chemotherapeutics, such as methotrexate and 5-fluorouracil.

An assay for functional complementation of the yeast gene met11 has been described (Shan et al., JBC, 274:32613-32618, 1999). In this assay, wildtype human MTHFR was shown to complement a metl 1 mutation in S. cerevisiae. However, this assay was not sensitive to quantitative changes in activity due to MTHFR mutations, as demonstrated by the similar ability of the functionally impaired allele A222V to complement the yeast mutation as compared to the wild-type enzyme; nor was this assay sensitive to the effects of folate availability.

In addition to folate utilizing enzymes, a handful of vitamin B₆- and B₁₂-dependent enzymes and enzymatic pathways are relevant to homocysteine metabolism, NTDs and other birth defects and adverse pregnancy outcomes. For example, defects in the B₆ utilizing enzyme cystathionine-β-synthase (“CBS”) lead to accumulation of homocysteine (Kraus et al., 1999. Cystathionine β-synthase mutations in homocystinuria. Hum Mut 13:362-375). As well, single nucleotide polymorphisms (“SNPs”) of the B₆ utilizing enzyme cystathionine-y-lyase (“CTH”) have also been associated with homocysteinemia (Wang et al., 2004. Single nucleotide polymorphism in CTH associated with variation in plasma homocysteine concentration. Clin Genet 65:483-486).

### SUMMARY OF INVENTION

The invention derives in part from the development of novel in vivo assays for identifying impaired alleles of enzyme-encoding genes within metabolic pathways and determining their sensitivity to cofactor remediation. Compound yeast mutants, comprising a first mutation allowing for complementation by a functionally homologous enzyme of interest, and a second mutation (or group of mutations) rendering the strain dependent upon supplementation with a cofactor, provide for the study of enzyme complementation as a function of cofactor availability. Cofactor-sensitive impaired alleles, including remediable alleles, may be identified and the cofactor-availability:enzyme-activity relationship may be analyzed using assays disclosed herein. The results obtained may be used to inform prophylactic and therapeutic nutrient supplementation approaches to prevent and treat conditions and diseases associated with metabolic enzyme dysfunction and aberrant metabolism.

The present invention also derives in part from the demonstration for the first time herein that cofactor remediation of low-frequency impaired alleles in enzyme-encoding genes is surprisingly common. As exemplified herein, multiple cofactor-sensitive genes in a metabolic pathway can each have multiple low frequency mutations in the population. Taken together, these mutations collectively have a more significant impact on the metabolic pathway than would be apparent from examination of a single low frequency impaired allele of a single gene. Moreover, since cells heterozygous for a plurality of such low frequency impaired alleles display quantitative defects, the aggregate frequencies of such individually rare alleles may contribute to common phenotypes even in the absence of more common polymorphism(s). Such low-frequency impaired alleles having impact on the pathway may also contribute to the phenotypic variation that is observed with common polymorphisms. Accordingly, the present invention contemplates diagnostic and prognostic methods focused in particular on the detection and characterization of such low frequency impaired alleles in enzyme-encoding genes, and determination of their effective remediation.

The present invention also derives in part from the specific application of these assays to identify and characterize novel low frequency impaired alleles in enzyme-encoding genes involved in folate/homocysteine metabolism in particular. As demonstrated herein with respect to MTHFR, a number of low-frequency impaired alleles exist that can cumulatively contribute to enzyme deficiency but can also be resolved by cofactor supplementation. The invention also derives in part from the finding that impaired alleles of MTHFR comprise sequence changes that map to the coding sequence of the N-terminal catalytic domain of the enzyme.

The invention therefore provides novel in vivo assays for detecting impaired but remediable alleles of enzyme-encoding genes involved in folate/homocysteine metabolism including, e.g., ATIC, GART, MAT1A, MAT2A, MTHFR, and MTHFS. Although the prior art describes a complementation assay in which wildtype human MTHFR activity complemented met11 deficiency (Shan et al. JBC, 274:32613-32618, 1999), this assay was not highly sensitive and could not detect all functionally impaired human MTHFR alleles. For example, the assay was not capable of distinguishing between wildtype MTHFR and the functionally impaired common polymorphism A222V. Further, this assay revealed nothing about the relationship between folate levels and enzyme activity.

In contrast to the prior art, the presently disclosed in vivo assays are highly sensitive and capable of unmasking impaired alleles of genes involved in folate/homocysteine metabolism, as demonstrated herein with respect to MTHFR, while simultaneously determining the sensitivity thereof to folate. The alleles identified include low frequency alleles, dominant or codominant alleles that exhibit phenotypes as heterozygotes, alleles that are folate-sensitive, including alleles that are folate-remediable, and alleles which possess combinations of these characteristics. Importantly, these impaired alleles are associated with the risk of a variety of conditions and diseases, as well as the varied efficacy and toxicity of chemotherapeutic agents. The deficiency of these impaired alleles may not manifest as a condition, disease, or varied response to chemotherapy in some individuals due to the compensatory effect of folate availability. The ability to unmask functionally impaired alleles of MTHFR provides for methods of screening for a risk of such conditions and diseases, as well as for the potential therapeutic efficacy and toxicity of chemotherapeutics.

The invention also provides novel in vivo assays for detecting impaired alleles of CTH and CBS. The ability to unmask functionally impaired alleles of these genes similarly provides for methods of screening for risk of associated diseases and conditions.

Accordingly, in one aspect, the invention provides in vivo assays for detecting impaired alleles of enzyme-encoding genes in metabolic pathways, and determining their sensitivity to cofactors. The assays comprise the use of yeast strains that comprise a first mutation in a first gene that may be complemented by the wildtype enzyme-encoding gene, and a second mutation in a second gene (or group of genes) that renders the yeast strain dependent on supplementation with the cofactor (or precursor thereof) for an assayable phenotype related to function of the first gene.

The methods comprise (i) introducing into a yeast cell a test allele of an enzyme-encoding gene, wherein the yeast cell comprises a first mutation in a first gene that is functionally homologous to the enzyme-encoding gene, and a second mutation in a second gene (or group of genes) that renders the yeast cell dependent upon supplementation with a cofactor required for enzyme function, wherein the first mutation alters a measurable characteristic of the yeast related to the function of the first gene; (ii) supplementing the growth medium with the cofactor; and (iii) detecting less restoration of the measurable characteristic in the presence of the test allele than in the presence of the wildtype enzyme, thereby detecting incomplete complementation of the first gene mutation by the test allele and identifying the test allele as an impaired allele. By titrating the amount of supplemented cofactor, the sensitivity of the impaired allele to cofactor availability is determined.

In one embodiment, diploid yeast are used. The diploid yeast may be homozygous or heterozygous for a test allele. Diploid yeast may comprise a wildtype gene and a test allele. Diploid yeast may comprise a combination of test alleles.

In a preferred embodiment, the enzyme-encoding gene corresponds in sequence to a naturally occurring allele, or to a compilation of individual naturally occurring alleles. In a preferred embodiment, the enzyme-encoding gene comprises an allele of a human enzyme-encoding gene, or a compilation of individual human alleles.

In a preferred embodiment, the yeast is S. cerevisiae.

In one embodiment, the first yeast gene is met13 and the second yeast gene is fol3. Such a yeast strain may be used to determine the activity of MTHFR alleles, and the response thereof to folate status. Accordingly, in one embodiment, the invention provides in vivo assays for determining the activity of MTHFR alleles, which are further capable of determining activity as a function of folate status. In a preferred embodiment, the enzyme-encoding gene comprises a naturally occurring human MTHFR allele. In another preferred embodiment, the enzyme-encoding gene comprises a compilation of individual human MTHFR alleles.

In a preferred embodiment, the assay method comprises comparing the activity of an MTHFR allele of interest to that of wildtype MTHFR.

In a preferred embodiment, the assay method comprises titrating the amount of folate to determine whether an MTHFR enzyme is sensitive to folate availability.

In one embodiment, the yeast is diploid. In one embodiment, the diploid yeast is heterozygous with respect to the MTHFR allele being tested for complementation. In one embodiment, the diploid yeast comprises wildtype MTHFR and a mutant MTHFR allele.

In a preferred embodiment, the measured output of the assay is growth.

In one embodiment, the first yeast gene is ade16 or ade17 and the second yeast gene is fol3. Such a yeast strain may be used to determine the activity of bifunctional enzyme AICAR Transformylase and IMP Cyclohydrolase (ATIC) alleles, and the response thereof to folate status. Accordingly, in one embodiment, the invention provides in vivo assays for determining the activity of ATIC alleles, which are further capable of determining activity as a function of folate status. In a preferred embodiment, the enzyme-encoding gene comprises a naturally occurring human ATIC allele. In another preferred embodiment, the enzyme-encoding gene comprises a compilation of individual human ATIC alleles.

In one embodiment, the first yeast gene is ade7 and the second yeast gene is fol3. Such a yeast strain may be used to determine the activity of glycinamide ribonucleotide transformylase (GART) alleles, and the response thereof to folate status. Accordingly, in one embodiment, the invention provides in vivo assays for determining the activity of GART alleles, which are further capable of determining activity as a function of folate status. In a preferred embodiment, the enzyme-encoding gene comprises a naturally occurring human GART allele. In another preferred embodiment, the enzyme-encoding gene comprises a compilation of individual human GART alleles.

In one embodiment, the first yeast gene is sam1 or sam2 and the second yeast gene is fol3. Such a yeast strain may be used to determine the activity of methionine adenosyltransferase I, alpha (MAT1A) alleles, and the response thereof to folate status. Accordingly, in one embodiment, the invention provides in vivo assays for determining the activity of MAT1A alleles, which are further capable of determining activity as a function of folate status. In a preferred embodiment, the enzyme-encoding gene comprises a naturally occurring human MAT1A allele. In another preferred embodiment, the enzyme-encoding gene comprises a compilation of individual human MAT1A alleles.

In one embodiment, the first yeast gene is sam1 or sam2 and the second yeast gene is fol3. Such a yeast strain may be used to determine the activity of methionine adenosyltransferase II, alpha (MAT2A) alleles, and the response thereof to folate status. Accordingly, in one embodiment, the invention provides in vivo assays for determining the activity of MAT2A alleles, which are further capable of determining activity as a function of folate status. In a preferred embodiment, the enzyme-encoding gene comprises a naturally occurring human MAT2A allele. In another preferred embodiment, the enzyme-encoding gene comprises a compilation of individual human MAT2A alleles.

In one embodiment, the first yeast gene is fau1 and the second yeast gene is fol3. Such a yeast strain may be used to determine the activity of methenyltetrahydrofolate synthetase (MTHFS) alleles, and the response thereof to folate status. Accordingly, in one embodiment, the invention provides in vivo assays for determining the activity of MTHFS alleles, which are further capable of determining activity as a function of folate status. In a preferred embodiment, the enzyme-encoding gene comprises a naturally occurring human MTHFS allele. In another preferred embodiment, the enzyme-encoding gene comprises a compilation of individual human MTHFS alleles.

In another embodiment, the first yeast gene is cys3, and the second group of yeast genes is sextuple-delete sno1Δ sno2Δ sno3Δ snz1Δ snz2Δ snz3Δ. Such a yeast strain may be used to determine the activity of CTH alleles, and the response thereof to vitamin B₆ status. Accordingly, in one embodiment, the invention provides in vivo assays for determining the activity of CTH alleles, which are further capable of determining activity as a function of vitamin B₆ status. In a preferred embodiment, the enzyme-encoding gene comprises a naturally occurring human CTH allele. In another preferred embodiment, the enzyme-encoding gene comprises a compilation of individual human CTH alleles.

In another embodiment, the first yeast gene is cys4, and the second group of yeast genes is sextuple-delete sno1Δ sno2Δ sno3Δ snz1Δ snz2Δ snz3Δ. Such a yeast strain may be used to determine the activity of CBS alleles, and the response thereof to vitamin B₆ status. Accordingly, in one embodiment, the invention provides in vivo assays for determining the activity of CBS alleles, which are further capable of determining activity as a function of vitamin B₆ status. In a preferred embodiment, the enzyme-encoding gene comprises a naturally occurring human CBS allele. In another preferred embodiment, the enzyme-encoding gene comprises a compilation of individual human CBS alleles.

In one aspect, the invention provides yeast strains capable of detecting impaired alleles of genes involved in folate/homocysteine metabolism and the sensitivity thereof to cofactors.

In one embodiment, the invention provides yeast strains capable of detecting impaired alleles of enzyme-encoding genes selected from the group consisting of ATIC, GART, MAT1A, MAT2AMTHFR, and MTHFS, and determining the responsiveness thereof to folate. In preferred embodiments, the yeast comprises the respective mutations and additions described hereinabove for each such enzyme-encoding gene.

In one embodiment, the invention provides yeast strains capable of detecting impaired alleles of CTH and determining the responsiveness thereof to vitamin B₆.

In one embodiment, the invention provides yeast strains capable of detecting impaired alleles of CBS and determining the responsiveness thereof to vitamin B₆.

In one aspect, the invention provides methods for detecting an impaired allele of an enzyme-encoding gene in a metabolic pathway, such as, e.g. folate/homocysteine metabolism. In one embodiment, the impaired allele(s) are naturally-occurring in human ATICGART, MAT1A, MAT2A, MTHFR, and/or MTHFS. In one embodiment, the impaired allele is a CBS allele. In one embodiment, the impaired allele is a CTH allele. In preferred embodiments, the methods comprise detecting an impaired allele in a metabolic enzyme-encoding gene which has been shown to be cofactor-remediable using the in vivo assays and methods provided herein.

In another aspect, the invention provides methods for identifying and/or characterizing a metabolic enzyme deficiency in a subject, comprising obtaining a sample from the subject and detecting the presence or absence of a plurality of impaired alleles in said sample, wherein the presence of at least one impaired allele indicates that the subject is at risk of an enzyme deficiency. The plurality of impaired alleles may be from the same enzyme-encoding gene in the metabolic pathway, or may be alleles from multiple genes in the same pathway.

In preferred embodiments, one or more of the impaired alleles are low-frequency alleles, e.g., generally expressed in less than 4% of the general population, more generally in less than 3% of the general population, preferably less than 2.5% to 2%, and most preferably in less than 1% of the general population. In preferred embodiments, one or more of the impaired alleles are cofactor-remediable alleles. In particularly preferred embodiments, the cofactor-remediable impaired alleles are identified by the in vivo assays and methods provided herein.

In another aspect, methods for detecting a predisposition to a cofactor-dependent enzyme deficiency in a subject are provided, comprising obtaining a sample from the subject and detecting the presence or absence of a plurality of impaired alleles in said sample, wherein the presence of at least one impaired allele indicates that the subject may have a remediable enzyme deficiency. The plurality of impaired alleles may be from the same enzyme-encoding gene in the metabolic pathway, or may be alleles from multiple genes in the same pathway.

In preferred embodiments, one or more of the impaired alleles are low-frequency alleles, e.g., generally expressed in less than 4% of the general population, more generally in less than 3% of the general population, preferably less than 2.5% to 2%, and most preferably in less than 1% of the general population. In preferred embodiments, one or more of the impaired alleles are cofactor-remediable alleles. In particularly preferred embodiments, the cofactor-remediable impaired alleles are identified by the in vivo assays and methods provided herein.

The detection of specific alleles in samples is common in the art and any conventional detection protocol may be advantageously employed in the subject methods including protocols based on, e.g., hybridization, amplification, sequencing, RFLP analysis, and the like, as described herein. Also contemplated for use herein are protocols and/or materials developed in the future having particular utility in the detection of alleles in nucleic acid samples.

In a further aspect, methods for treating a metabolic enzyme deficiency in a subject are provided, comprising obtaining a sample from a subject having or suspected of having such a deficiency, detecting the presence or absence of a plurality of cofactor-remediable impaired alleles in the sample, and administering an appropriate cofactor supplement to the subject based on the number and type of impaired allele(s) detected in the sample, as described herein.

In one embodiment, the methods further comprise use of an in vivo assay for determining enzyme activity, as described herein.

In one embodiment, the methods further comprise use of an in vivo assay for determining enzyme activity, as described herein, and detecting a mutation in an enzyme-encoding nucleic acid.

In one embodiment, the methods further comprise use of an in vivo assay for determining enzyme activity, as described herein, and a temperature sensitivity assay to determine enzyme stability at an elevated temperature.

In one embodiment, the methods further comprise use of an in vivo assay for determining enzyme activity, as described herein, and an in vitro assay for determining the specific activity of the enzyme.

In one aspect, the invention provides methods of screening for risk of a disease or condition associated with aberrant homocysteine metabolism. The methods comprise screening for an impaired allele of a gene involved in homocysteine metabolism, as disclosed herein. In a preferred embodiment, the methods comprise detecting an impaired allele which has been characterized as such using an in vivo assay described herein. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birthweight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma In one embodiment, the methods comprise screening for an impaired allele of ATIC, GART, MAT1A, MAT2A, MTHFR, and/or MTHFS, as described herein.

In one embodiment, the methods comprise screening for an impaired allele of CBS, as described herein.

In one embodiment, the methods comprise screening for an impaired allele of CTH, as described herein.

In one aspect, the invention provides methods for determining the chemotherapeutic response potential of an individual. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in the individual by the in vivo assay methods described herein and/or by application of detection methods for specific alleles indicates a decreased response potential.

In one aspect, the invention provides methods of determining potential chemotherapeutic toxicity for an individual. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in the individual by the in vivo assay methods described herein and/or by application of detection methods for specific alleles indicates an increased toxicity potential.

In one aspect, the invention provides isolated nucleic acids corresponding in sequence to alleles of an enzyme-encoding gene selected from the group consisting of MTHFR ATIC, MTHFS, MAT1A, MAT2A, and GART. In one embodiment, the isolated nucleic acid has and/or comprises a sequence of an allele of an MTHFR gene, e.g., a SNP disclosed in Table A. In one embodiment, the isolated nucleic acid has and/or comprises a sequence of an allele of an ATIC gene, e.g., a SNP disclosed in Table B. In one embodiment, the isolated nucleic acid has and/or comprises a sequence of an allele of an MTHFS gene, e.g., a SNP disclosed in Table C. In one embodiment, the isolated nucleic acid has and/or comprises a sequence of an allele of an MAT1A gene, e.g., a SNP disclosed in Table D. In one embodiment, the isolated nucleic acid has and/or comprises a sequence of an allele of an MAT2A gene, e.g., a SNP disclosed in Table E. In one embodiment, the isolated nucleic acid has and/or comprises a sequence of an allele of an GART gene, e.g., a SNP disclosed in Table F. In one embodiment, the nucleic acid corresponds to a sequence of an MTHFR allele and comprises a sequence encoding a non-synonymous mutation in the MTHFR protein selected from the group consisting of M110I, H213R, D223N, D291N, R519C, R519L, and 0648P.

In one aspect, the invention provides arrays for detecting impaired alleles of genes involved in folate/homocysteine metabolism.

In one embodiment, the invention provides arrays for detecting an impaired allele of a gene selected from the group consisting of ATIC, GART, MAT1A, MAT2A, MTHFR and MTHFS. In a preferred embodiment, the array is capable of detecting more than one impaired allele for a gene selected from the group. In a preferred embodiment, the array is capable of detecting more than one impaired allele a plurality of genes selected from the group. In one embodiment, the array is capable of detecting more than one impaired allele from each of a plurality of genes selected from the group. In a preferred embodiment, the array is capable of detecting such an impaired allele that is a remediable impaired allele. In a preferred embodiment, the array is capable of detecting a plurality of such impaired alleles that are remediable impaired alleles. In preferred embodiments, at least one of the impaired alleles is a low-frequency allele.

In one embodiment, the invention provides arrays for detecting an impaired MTHFR allele. In one embodiment, the array comprises one or more nucleic acids capable of hybridizing to an MTHFR allele comprising a non-synonymous mutation selected from the group consisting of those encoding M110I, H213R, D223N, D291N, R519C, R519L, and Q648P.

In one embodiment, the invention provides arrays for detecting impaired alleles of CBS. The arrays comprise one or more nucleic acids capable of hybridizing to an impaired allele of CBS.

In one embodiment, the invention provides arrays for detecting impaired alleles of CTH. The arrays comprise one or more nucleic acids capable of hybridizing to an impaired allele of CTH.

In a preferred embodiment, the invention provides arrays for detecting impaired alleles of a plurality of genes involved in folate/homocysteine metabolism. The arrays of the invention may use any of the many array, probe and readout technologies known in the art.

In one aspect, the invention provides a method of preventing a condition or disease associated with aberrant folate/homocysteine metabolism in an individual harboring a remediable impaired allele of a gene involved in folate/homocysteine metabolism. In one embodiment, the method comprises increasing the individual's intake of folate. In one embodiment, the method comprises increasing the individual's intake of vitamin B₆. In a preferred embodiment, the method comprises a method of screening for risk of a disease or condition associated with aberrant folate/homocysteine metabolism, as described herein.

In one aspect, the invention provides a method of treating a condition or disease associated with aberrant folate/homocysteine metabolism wherein the patient harbors a remediable impaired allele of a gene involved in folate/homocysteine metabolism. In one embodiment, the method comprises increasing the patient's intake of folate. In one embodiment, the method comprises increasing the individual's intake of vitamin B₆. In a preferred embodiment, the method comprises a method of screening for risk of a disease or condition associated with aberrant folate/homocysteine metabolism, as described herein.

In one aspect, the invention provides a method of increasing the chemotherapeutic response potential of an individual harboring a remediable impaired allele of a gene involved in folate/homocysteine metabolism. The method comprises increasing the individual's intake of folate. In a preferred embodiment, the method comprises a method of screening for risk of a disease or condition associated with aberrant folate/homocysteine metabolism, as described herein. In a preferred embodiment, the gene is selected from the group consisting of MTHFR, ATIC, MTHFS, MAT1A, MAT2A, and GART.

In one aspect, the invention provides a method of decreasing the toxicity of a chemotherapeutic for an individual harboring a remediable impaired allele of a gene involved in folate/homocysteine metabolism. The method comprises increasing the individual's intake of folate. In a preferred embodiment, the method comprises a method of screening for risk of a disease or condition associated with aberrant folate/homocysteine metabolism, as described herein. In a preferred embodiment, the gene is selected from the group consisting of MTHFR, ATIC, MTHFS, MAT1A, MAT2A, and GART.

### BRIEF DESCRIPTION OF THE DRAWINGS

FIG. 1. Effects of folinic acid supplementation on growth rate of fol3Δ::KanMX cells and cellular activity of human MTHFR. (a) Growth of fol3Δ::KanMX MET13 haploid yeast was measured in 96-well plates as described in Materials and Methods. Media was supplemented with folinic acid at the indicated concentrations. The curve labeled FOL3 (FOL3 MET13) was from growth in medium without folinic acid. (b) Growth of fol3Δ::KanMX met13Δ::KanMX haploid yeast transformed with phMTHFR in media lacking methionine and supplemented with folinic acid at the indicated concentrations. 3 independent transformants were tested at each folinic acid concentration to test reproducibility. The curve labeled met13Δ represented a single isolate of cells, transformed with empty vector, grown at 50 μg/ml folinic acid.

FIG. 2. Functional impact and folate-remediability of nonsynonymous MTHFR population variants. (a) 6 MTHFR variants were tested for the ability to rescue fol3Δ::KanMX met13Δ::KanMX cells in media lacking methionine at 3 different folinic acid concentrations. The M110I allele and the M1101 A222V doubly-substituted allele were tested only at 50 and 25 μg/ml folinic acid. The curve labeled Major corresponds to the most common MTHFR allele in the population. Each curve is from a pool of 3-6 independent transformants. (b) Schematic of the MTHFR protein (656 amino acids) divided into a N-terminal catalytic domain and a C-terminal regulatory domain of nearly equal size (35). Positions of all nonsynonymous changes are indicated. Benign changes are in green. Changes numbered 1 through 4 represent folate-remedial alleles indicated in increasing order of severity. Change #5 (R134C) was nearly loss-of-function and not designated as folate-remedial (see Results) but was somewhat folate-augmentable.

FIG. 3. Enzyme activity of MTHFR variants. Crude yeast extract from cells transformed with the indicated MTHFR constructs was prepared and assayed for MTHFR activity as described herein. Heat treatment for the indicated times was done on reactions prior to addition of radiolabeled substrate. Measurements were averages of two independent sets of triplicate assays; error bars are standard deviation for the 6 data points.

FIG. 4. Heterozygote phenotypes for MTHFR variants as recapitulated in yeast. Homozygosity or heterozygosity of MTHFR alleles was recreated in diploid yeast for the major, R134C and A222V alleles as described herein. Diploids were obtained from the mating of haploid strains that each expressed a single allele of MTHFR integrated in the genome. Growth as a function of folinic acid supplementation was assayed exactly as for haploids.

FIG. 5. Immunoblot of human MTHFR variants expressed in yeast. (a) Extracts were made from yeast cells carrying different MTHFR alleles and detected with anti-HA antibody as described herein. A222V M110I was a doubly substituted allele; Major indicates the most common MTHFR allele in the population. The two right-most lanes were, side-by-side, the major allele and the non-phosphorylatable T34A allele (37). (b) The ratio of signal intensities of the unphosphorylated lower band to the phosphorylated upper band for all variants of MTHFR identified in this study plotted as a function of increasing severity of functional impact. Alleles on the x-axis were classified as benign or rank-ordered with respect to activity. All benign alleles (including the Major allele and all regulatory domain changes) were plotted and show nearly identical ratios of the two MTHFR species, thus the symbols overlapped.

FIG. 6. Assays for B₆ (pyridoxine)-responsiveness in two human B₆ enzymes: CBS and CTH.

### DETAILED DESCRIPTION OF THE INVENTION

As indicated above, the present invention provides novel in vivo assays for identifying impaired alleles of enzyme-encoding genes within metabolic pathways and determining their sensitivity to cofactor remediation. Compound yeast mutants, comprising a first mutation allowing for complementation by a functionally homologous enzyme of interest, and a second mutation (or group of mutations) rendering the strain dependent upon supplementation with a cofactor, provide for the study of enzyme complementation as a function of cofactor availability. Significantly, the present invention also demonstrates that cofactor remediation of low-frequency impaired alleles in enzyme-encoding genes is surprisingly common, and that these alleles can collectively have a significant impact on the metabolic pathway. Accordingly, the present invention contemplates diagnostic and prognostic methods focused in particular on the detection and characterization of such low-frequency impaired alleles in enzyme-encoding genes, and determination of their effective remediation.

The “N-terminal catalytic domain” of MTHFR refers to amino acids 1-359 in human MTHFR. The reference human MTHFR mRNA sequence is found at Genbank accession no. NM_{—}005957, while the encoded 656 amino acid sequence is found at Genbank accession no. NP_{—}005958.

By MTHFR dysfunction is meant a deviation from wildtype MTHFR activity. Enzyme dysfunction and associated conditions and diseases can arise through, for example, changes in the specific activity of an enzyme, mislocalization of an enzyme, changes in the level of an enzyme, and other changes.

In vivo assays for measuring enzyme activity and sensitivity thereof to cofactors

The assays provided herein may be used to test the ability of alleles of genes encoding enzymes to complement mutations in functionally homologous yeast genes, as well to measure the responsiveness of these enzymes to cofactors. The assays comprise measuring an output, or phenotype, that is associated with normal function of the yeast gene and altered by its dysfunction.

The assays comprise the use of yeast strains that comprise a first mutation allowing for complementation by a functionally homologous enzyme of interest, and a second mutation rendering the strain dependent upon supplementation with cofactor for an assayable phenotype related to function of the first gene.

The methods comprise (i) introducing into a yeast cell a test allele of an enzyme-encoding gene, wherein the yeast cell comprises a first mutation in a first gene that is functionally homologous to the enzyme-encoding gene, and a second mutation in a second gene (or group of genes) that renders the yeast cell dependent upon supplementation with a cofactor required for enzyme function, wherein the first mutation alters a measurable characteristic of the yeast related to the function of the first gene; (ii) supplementing the growth medium with the cofactor; and (iii) detecting less restoration of the measurable characteristic in the presence of the test allele than in the presence of the wildtype enzyme, thereby detecting incomplete complementation of the first gene mutation by the test allele and identifying the test allele as an impaired allele. By varying the amount of supplemented cofactor, the sensitivity of the impaired allele to cofactor availability is determined.

In a preferred embodiment, the test allele of an enzyme-encoding gene corresponds in sequence to a naturally occurring allele, or to a compilation of individual naturally occurring polymorphisms. In a preferred embodiment, the test allele corresponds in sequence to an allele of a human gene, or to a compilation of individual polymorphisms in a plurality of human alleles.

In a preferred embodiment, the yeast is Saccharomyces cerevisiae (“S. cerevisiae”), though other species of yeast may be used.

In one embodiment, diploid yeast are used. The diploid yeast may be homozygous or heterozygous for a test allele. Diploid yeast may comprise a wildtype gene and a test allele. Diploid yeast may comprise a combination of test alleles. As demonstrated herein, functionally impaired alleles may include alleles having a heterozygous phenotype. In one embodiment, the diploid yeast is heterozygous with respect to the allele being tested for complementation. In one embodiment, the diploid yeast comprises a wildtype allele and an impaired allele of an enzyme-encoding gene.

In a preferred embodiment, the measured output of the assay is growth.

In a preferred embodiment, the assay method comprises comparing the activity of a test allele of interest to that of a corresponding wildtype allele.

In one embodiment, the invention provides in vivo assays for determining the activity of a test allele, e.g., an allele of an enzyme-encoding gene. In one embodiment, the enzyme-encoding gene is involved in or related to folate/homocysteine metabolism. In another embodiment, the test allele is selected from the group consisting of an MTHFR allele, ATIC allele, GART allele, an MAT1A allele, an MAT2A allele, and an MTHFS allele, which assays are further capable of determining activity as a function of folate status. In another embodiment, the enzyme-encoding allele is selected from the group consisting of a CTH allele and CBS allele.

In one embodiment, the test allele is an MTHFR allele and comprises at least one substitution in the N-terminus catalytic domain and at least one mutation in the C-terminus regulatory region. While substitutions in the C-terminus region alone do not typically impair function, they may combine with other substitutions to functionally impair an allele.

In a preferred embodiment, the first mutation is in the yeast gene met13, which may be functionally complemented by wildtype human MTHFR. In another embodiment, the first yeast gene is ade16 or ade17, which may be functionally complemented by wildtype human ATIC. In one embodiment, the first yeast gene is ade7, which may be functionally complemented by wildtype human GART. In one embodiment, the first yeast gene is sam1 or sam2, which may be functionally complemented by wildtype human MAT1A or wildtype human MAT2A. In one embodiment, the first yeast gene is fau1, which may be functionally complemented by wildtype human MTHFS.

In a preferred embodiment, the second mutation is in the yeast gene fol3, which renders the yeast dependent upon folate in supplemented medium. Such a yeast strain may be used to determine the activity of a test allele, the test allele depending on the first mutation, and the response thereof to folate status. For example, a compound yeast having a first mutation in the yeast gene metl, and a second mutation in the yeast gene fol3, may be used to determine the activity of an MTHFR allele and the response thereof to folate status.

In a preferred embodiment, the assay method comprises varying the amount of folate to determine whether the enzyme encoded by the test allele is sensitive to folate availability. In a preferred embodiment, the assay method includes measuring output in the presence of less than 50 μg/ml folate. In a preferred embodiment, the assay method includes measuring output in the presence of about 50 μg/ml folate. In a preferred embodiment, the assay method includes measuring output in the presence of more than 50 μg/ml folate.

In one embodiment, the folate is varied to determine whether an impaired allele of an enzyme-encoding gene is remediable by folate.

In another embodiment, the first yeast gene is cys3, and the second yeast gene is sextuple-delete sno1Δ sno2Δ sno3Δ snz1Δ snz2Δ snz3Δ. Such a yeast strain may be used to determine the activity of CTH alleles, and the response thereof to vitamin B₆ status. Accordingly, in one embodiment, the invention provides in vivo assays for determining the activity of CTH alleles, which are further capable of determining activity as a function of vitamin B₆ status. In a preferred embodiment, the CTH allele comprises a naturally occurring human allele. In another preferred embodiment, the CTH allele comprises a compilation of individual human CTH alleles.

In another embodiment, the first yeast gene is cys4, and the second yeast gene is sextuple-delete sno1Δ sno2Δ sno3Δ snz1Δ snz2Δ snz3Δ. Such a yeast strain may be used to determine the activity of CBS alleles, and the response thereof to vitamin B₆ status. Accordingly, in one embodiment, the invention provides in vivo assays for determining the activity of CBS alleles, which are further capable of determining activity as a function of vitamin B₆ status. In a preferred embodiment, the CBS allele comprises a naturally occurring human allele. In another preferred embodiment, the CBS allele comprises a compilation of individual human CBS alleles.

Table 1 below lists enzyme-encoding genes and provides exemplary compound yeast mutations that may be used to determine the activity of an allele of the enzyme-encoding gene.

| HGNC | Yeast Screening Strain Backgrounds |
|---|---|
| ATIC | fol3 ade16 ade17 |
| CBS | sno/snz1 sno/snz2 sno/snz3 cys4 |
| CTH | sno/snz1 sno/snz2 sno/snz3 cys3 |
| GART | fol3 ade8 |
| MAT1A | fol3 sam1 sam2 |
| MAT2A | fol3 sam1 sam2 |
| MTHFR | fol3 met13 |
| MTHFS | fol3 fau1 |

Yeast strains may be generated by methods well known in the art. For example, see Shan et al., JBC, 274:32613-32618, 1999.

Introduction of nucleic acids into yeast strains may be done using methods well known in the art. For example, see Shan et al., JBC, 274:32613-32618, 1999.

Novel Alleles of Enzyme-Encoding Genes

As described in the Examples section, single nucleotide polymorphisms that subtly affect enzymes, e.g., that result in an impaired allele of an enzyme-encoding gene may be characterized using the in vivo assay disclosed herein regardless of the frequency of the allele. For example, the methods disclosed herein were used to determine whether an allele is an impaired allele, and if so, whether the impaired allele is cofactor-remediable. Provided in Table 3 and Tables A-F are single nucleotide polymorphisms for the enzyme-encoding genes MTHFR, ATIC, MTHFS, MAT1A, MAT2A and GART that have been characterized (Table 3) or may be characterized (Tables A-F) by the assay described herein. These tables also provide SNPs for these genes which have not been previously identified. Accordingly, disclosed herein are novel alleles for an enzyme-encoding gene selected from the group consisting of MTHFR, ATIC, MTHFS, MAT1A, MAT2A, and GART. These alleles may be characterized using the assay disclosed herein, and may be advantageously detected in the methods of screening, preventing and treating as disclosed herein. An ordinarily skilled artisan will recognize and appreciate that characterization of an impaired allele as cofactor remediable informs the methods of screening, preventing and treating as disclosed herein.

As used herein, an “allele” is a nucleotide sequence, such as a single nucleotide polymorphism (SNP), present in more than one form in a genome. An “allele” as used herein is not limited to the naturally occurring sequence of a genomic locus. “Allele” includes transcripts and spliced sequence derived therefrom (e.g., mRNA sequence, cDNA sequence). An “allele” may be a naturally occurring allele or a synthetic allele. These may include mutations in the N-terminal catalytic domain as well as mutations in the C-terminal regulatory region.

“Homozygous”, according to the present invention, indicates that the two copies of the gene or SNP are identical in sequence to the other allele. For example, a subject homozygous for the wild-type allele of an enzyme-encoding gene contains at least two identical copies of the sequence. Such a subject would not be predisposed to a cofactor-dependent enzyme deficiency within a metabolic pathway.

“Heterozygous,” as used herein, indicates that two different copies of the allele are present in the genome, for example one copy of the wild-type allele and one copy of the variant allele, which may be an impaired allele. A subject having such a genome is heterozygous, and may be predisposed to a cofactor-dependent enzyme deficiency within a metabolic disease. “Heterozygous” also encompasses a subject having two different mutations in its alleles.

By “impaired allele” is meant an allele of a gene encoding a metabolic enzyme that is functionally impaired, which functional impairment may or may not be cofactor-remediable.

An “impaired aliele mutation” refers to the particular nucleic acid mutation that underlies functional impairment of an impaired allele and distinguishes an impaired aliele from wildtype sequence at the location of the mutation. Typically, an impaired allele mutation is a non-synonymous point mutation in a single codon.

“Cofactor-remediable” refers to the ability of altered cofactor level to compensate for the functional impairment of an impaired metabolic enzyme.

Supplementation with a cofactor includes supplementation with a precursor of a cofactor that may be converted to the cofactor.

“Cofactor” refers to factors that are direct cofactors of enzymes of interest (e.g., folate for MTHFR, ATIC, GART, MAT1A, MAT2A, and MTHFS) as well as factors that are indirect cofactors for enzymes of interest. Thus, cofactors can directly or indirectly impact enzyme function.

Measures of frequency known in the art include “aliele frequency”, namely the fraction of genes in a population that have a specific SNP. The allele frequencies for any gene should sum to 1. Another measure of frequency known in the art is the “heterozygote frequency” namely, the fraction of individuals in a population who carry two alleles, or two forms of a SNP of a gene, one inherited from each parent. Alternatively, the number of individuals who are homozygous for a particular allele of a gene may be a useful measure. The relationship between allele frequency, heterozygote frequency, and homozygote frequency is described for many genes by the Hardy-Weinberg equation, which provides the relationship between aliele frequency, heterozygote frequency and homozygote frequency in a freely breeding population at equilibrium. Most human variances are substantially in Hardy-Weinberg equilibrium. As used herein, a “low frequency aliele” has an aliele frequency of less than 4%.

Disclosed herein are novel alleles for human enzyme-encoding genes involved in or relevant to folate/homocystein metabolism. By “folate/homocysteine metabolism” is meant folate and/or homocysteine metabolism. Such enzyme-encoding genes include MTHFR, ATIC, GART, MAT1A, MAT2A, MTHFS. The Hugo Gene Nomenclature Committee (HGNC) symbols, GenelDs, NCBI nucleotide accession numbers (NC_), NCBI polypeptide accession numbers (NB_) and names of enzyme-encoding genes involved in or relevant to folate/homocysteine metabolism is provided in Table 2.

TABLE 2

| | | NCBI | NCBI | |
|---|---|---|---|---|
| HGNC | GeneID | nucleotide | polypeptide | Name |
| ATIC | 471 | NC_000002.10 | NM_004044 | aminoimidazole-4-carboxamide |
| | | | | ribonucleotide |
| | | | | formyltransferase/IMP |
| | | | | cyclohydrolase |
| GART | 2618 | NC_000021.7 | NM_000819 | glycinamide ribonucleotide |
| | | | | transformylase |
| MAT1A | 4143 | NC_000010.9 | NM_000429 | methionine adenosyltransferase I, |
| | | | | alpha |
| MAT2A | 4144 | NC_000002.10 | NM_005911 | methionine adenosyltransferase II, |
| | | | | alpha |
| MTHFR | 4524 | NC_000001.9 | NM_005957 | methylenetetrahydrofolate reductase |
| MTHFS | 10588 | NC_000015.8 | NM_006441 | methenyltetrahydrofolate synthetase |

In one aspect, the invention provides isolated nucleic acids corresponding in sequence to novel human enzyme-encoding alleles involved in folate/homocisteine metabolism. For example, the invention provides isolated nucleic acids corresponding in sequence to an enzyme-encoding allele selected from the group consisting of an MTHFR allele, a ATIC allele, a GART allele, an MAT1A allele, an MAT2A allele, and an MTHFS allele, which may or may not be cofactor-remediable. These novel alleles include low frequency alleles. These novel alleles include impaired alleles.

Accordingly, provided herein is an isolated nucleic acid corresponding in sequence to an allele of an MTHFR gene, wherein said nucleic acid comprises a SNP found at a nucleotide selected from the group consisting of nucleotide 4078 of the MTHFR gene; nucleotide 4234 of the MTHFR gene; nucleotide 5733 of the MTHFR gene; nucleotide 5872 of the MTHFR gene; nucleotide 6642 of the MTHFR gene; nucleotide 6657 of the MTHFR gene; nucleotide 6681 of the MTHFR gene; nucleotide 6774 of the MTHFR gene; nucleotide 10906 of the MTHFR gene; nucleotide 11656 of the MTHFR gene; nucleotide 11668 of the MTHFR gene; nucleotide 11902 of the MTHFR gene; nucleotide 12232 of the MTHFR gene; nucleotide 2622 of the MTHFR gene; nucleotide 12759 of the MTHFR gene; nucleotide 13040 of the MTHFR gene; nucleotide 14593 of the MTHFR gene; nucleotide 14612 of the MTHFR gene; nucleotide 14705 of the MTHFR gene; nucleotide 13170 of the MTHFR gene; nucleotide 116401 of the MTHFR gene; and nucleotide 116451 of the MTHFR gene. The sequences of the SNPs at these positions is provided in Table A.

Also provided herein is an isolated nucleic acid corresponding in sequence to an allele of an ATIC gene, wherein said nucleic acid comprises a SNP found at a nucleotide selected from the group consisting of nucleotide 1100 of the ATIC gene; nucleotide 1114 of the ATIC gene; nucleotide 1179 of the ATIC gene; nucleotide 1244 of the ATIC gene; nucleotide 1270 of the ATIC gene; nucleotide 1288 of the ATIC gene; nucleotide 1301 of the ATIC gene; nucleotide 1380 of the ATIC gene; nucleotide 1396 of the ATIC gene; nucleotide 1453 of the ATIC gene; nucleotide 1506 of the ATIC gene; nucleotide 1689 of the ATIC gene; nucleotide 7227 of the ATIC gene; nucleotide 7232 of the ATIC gene; nucleotide 7388 of the ATIC gene; nucleotide 8756 of the ATIC gene; nucleotide 8808 of the ATIC gene; nucleotide 14099 of the ATIC gene; nucleotide 14140 of the ATIC gene; nucleotide 14144 of the ATIC gene; nucleotide 14183 of the ATIC gene; nucleotide 14229 of the ATIC gene; nucleotide 14238 of the ATIC gene; nucleotide 14245 of the ATIC gene; nucleotide 14260 of the ATIC gene; nucleotide 14489 of the ATIC gene; nucleotide 14970 of the ATIC gene; nucleotide 15003 of the ATIC gene; nucleotide 15040 of the ATIC gene; nucleotide 15043 of the ATIC gene; nucleotide 15149 of the ATIC gene; nucleotide 15240 of the ATIC gene; nucleotide 15844 of the ATIC gene; nucleotide 16063 of the ATIC gene; nucleotide 21363 of the ATIC gene; nucleotide 21372 of the ATIC gene; nucleotide 21400 of the ATIC gene; nucleotide 21521 of the ATIC gene; nucleotide 21611 of the ATIC gene; nucleotide 22187 of the ATIC gene; nucleotide 22273 of the ATIC gene; nucleotide 22282 of the ATIC gene; nucleotide 22291 of the ATIC gene; nucleotide 22342 of the ATIC gene; nucleotide 22512 of the ATIC gene; nucleotide 22519 of the ATIC gene; nucleotide 22538 of the ATIC gene; nucleotide 22564 of the ATIC gene; nucleotide 22589 of the ATIC gene; nucleotide 22737 of the ATIC gene; nucleotide 24992 of the ATIC gene; nucleotide 25009 of the ATIC gene; nucleotide 27757 of the ATIC gene; nucleotide 27855 of the ATIC gene; nucleotide 27985 of the ATIC gene; nucleotide 28015 of the ATIC gene; nucleotide 33901 of the ATIC gene; nucleotide 33919 of the ATIC gene; nucleotide 33920 of the ATIC gene; nucleotide 33933 of the ATIC gene; nucleotide 35723 of the ATIC gene; nucleotide 35737 of the ATIC gene; nucleotide 35742 of the ATIC gene; nucleotide 35840 of the ATIC gene; nucleotide 35917 of the ATIC gene; nucleotide 35968 of the ATIC gene; nucleotide 35973 of the ATIC gene; nucleotide 38338 of the ATIC gene; nucleotide 38342 of the ATIC gene; nucleotide 38437 of the ATIC gene; nucleotide 38342 of the ATIC gene; nucleotide 38582 of the ATIC gene; nucleotide 38627 of the ATIC gene; nucleotide 38667 of the ATIC gene; and nucleotide 38725 of the ATIC gene. The sequences of the SNPs at these positions is provided in Table B.

Also provided herein is an isolated nucleic acid corresponding in sequence to an allele of an MTHFS gene, wherein said nucleic acid comprises a SNP found at a nucleotide selected from the group consisting of nucleotide 8808 of the MTHFS gene; nucleotide 8912 of the MTHFS gene; nucleotide 8957 of the MTHFS gene; nucleotide 8998 of the MTHFS gene; nucleotide 52560 of the MTHFS gene; nucleotide 52878 of the MTHFS gene; and nucleotide 52902 of the MTHFS gene. The sequences of the SNPs at these positions is provided in Table C.

Also provided herein is an isolated nucleic acid corresponding in sequence to an allele of an MAT1A gene, wherein said nucleic comprises a SNP found at a nucleotide selected from the group consisting of nucleotide 5045 of the MAT1A gene; nucleotide 5181 of the MAT1A gene; nucleotide 5233 of the MAT1A gene; nucleotide 6739 of the MAT1A gene; nucleotide 6795 of the MAT1A gene; nucleotide 9833 of the MAT1A gene; nucleotide 10006 of the MAT1A gene; nucleotide 10312 of the MAT1A gene; nucleotide 10339 of the MAT1A gene; nucleotide 10374 of the MAT1A gene; nucleotide 10484 of the MAT1A gene; nucleotide 10555 of the MAT1A gene; nucleotide 14038 of the MAT1A gene; nucleotide 14114 of the MAT1A gene; nucleotide 14177 of the MAT1A gene; nucleotide 15424 of the MAT1A gene; nucleotide 15500 of the MAT1A gene; nucleotide 15646 of the MAT1A gene; nucleotide 15706 of the MAT1A gene; nucleotide 15715 of the MAT1A gene; nucleotide 15730 of the MAT1A gene; nucleotide 15758 of the MAT1A gene; nucleotide 16133 of the MAT1A gene; nucleotide 16174 of the MAT1A gene; nucleotide 15706 of the MAT1A gene; nucleotide 15715 of the MAT1A gene; nucleotide 15730 of the MAT1A gene; nucleotide 15758 of the MAT1A gene; nucleotide 16133 of the MAT1A gene; nucleotide 16174 of the MAT1A gene; nucleotide 16218 of the MAT1A gene; and nucleotide 16971 of the MAT1A gene. The sequences of the SNPs at these positions is provided in Table D.

Also provided herein is an isolated nucleic acid corresponding in sequence to an allele of an MAT2A gene, wherein said nucleic acid comprises a SNP found at a nucleotide selected from the group consisting of nucleotide 2871 of the MAT2A gene; nucleotide 2873 of the MAT2A gene; nucleotide 2939 of the MAT2A gene; nucleotide 3287 of the MAT2A gene; nucleotide 3394 of the MAT2A gene; nucleotide 3466 of the MAT2A gene; nucleotide 3498 of the MAT2A gene; nucleotide 3650 of the MAT2A gene; nucleotide 3704 of the MAT2A gene; nucleotide 4174 of the MAT2A gene; nucleotide 4449 of the MAT2A gene; nucleotide 4476 of the MAT2A gene; nucleotide 4608 of the MAT2A gene; nucleotide 4660 of the MAT2A gene; nucleotide 4692 of the MAT2A gene; nucleotide 4931 of the MAT2A gene; nucleotide 5313 of the MAT2A gene; nucleotide 5460 of the MAT2A gene; and nucleotide 5480 of the MAT2A gene. The sequences of the SNPs at these positions is provided in Table E.

Also provided herein is an isolated nucleic acid corresponding in sequence to an allele of a GART gene, wherein said nucleic acid comprises a one SNP found at a nucleotide in the GART gene selected from the group consisting of nucleotide 3782 of the GART gene; nucleotide 3842 of the GART gene; nucleotide 7745 of the GART gene; nucleotide 7984 of the GART gene; nucleotide 10775 of the GART gene; nucleotide 11521 of the GART gene; nucleotide 11522 of the GART gene; nucleotide 11541 of the GART gene; nucleotide 12356 of the GART gene; nucleotide 14200 of the GART gene; nucleotide 14273 of the GART gene; nucleotide 14282 of the GART gene; nucleotide 14739 of the GART gene; nucleotide 14781 of the GART gene; nucleotide 18055 of the GART gene; nucleotide 18064 of the GART gene; nucleotide 18130 of the GART gene; nucleotide 18142 of the GART gene; nucleotide 18197 of the GART gene; nucleotide 18232 of the GART gene; nucleotide 18401 of the GART gene; nucleotide 20812 of the GART gene; nucleotide 20825 of the GART gene; nucleotide 16174 of the GART gene; nucleotide 15706 of the GART gene; nucleotide 20862 of the GART gene; nucleotide 22481 of the GART gene; nucleotide 22521 of the GART gene; nucleotide 25425 of the GART gene; nucleotide 25433 of the GART gene; nucleotide 25601 of the GART gene; nucleotide 25867 of the GART gene; nucleotide 25912 of the GART gene; nucleotide 25951 of the GART gene; nucleotide 25956 of the GART gene; nucleotide 26127 of the GART gene; nucleotide 26195 of the GART gene; nucleotide 31627 of the GART gene; nucleotide 31641 of the GART gene; nucleotide 31887 of the GART gene; nucleotide 31902 of the GART gene; nucleotide 31933 of the GART gene; nucleotide 33173 of the GART gene; nucleotide 33264 of the GART gene; nucleotide 31933 of the GART gene; nucleotide 33173 of the GART gene; nucleotide 33264 of the GART gene; nucleotide 33286 of the GART gene; nucleotide 36963 of the GART gene; nucleotide 36964 of the GART gene; nucleotide 37428 of the GART gene; nucleotide 37433 of the GART gene; nucleotide 38762 of the GART gene; nucleotide 38914 of the GART gene; and nucleotide 38989 of the GART gene. The sequences of the SNPs at these positions is provided in Table F.

In one embodiment, the invention provides isolated nucleic acids corresponding in sequence to human MTHFR alleles comprising a sequence encoding a non-synonymous mutation in the MTHFR protein selected from the group consisting of M110I, H213R, D223N, D291N, R519C, R519L, and Q648P. In one embodiment, the invention provides nucleic acids corresponding in sequence to two or more human MTHFR alleles comprising a sequence encoding a non-synonymous mutation in the MTHFR protein selected from the group consisting of M110I, H213R, D223N, D291N, R519C, R519L, and Q648P.

The term “isolated” as used herein includes polynucleotides substantially free of other nucleic acids, proteins, lipids, carbohydrates or other materials with which it is naturally associated. Polynucleotide sequences of the invention include DNA and RNA sequences.

The nucleic acids provided herein may be useful as probes (e.g., allele specific oligonucleotide probes) or primers in the methods of detecting disclosed herein. The design of appropriate probes or primers for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as an allele specific oligonucleotide probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCI at temperatures of about 50° C. to about 70° C. Such selective conditions may tolerate little, if any, mismatch between the probe and the template or target polynucleotide fragments.

Also provided are vectors comprising nucleic acids of the invention. These vectors include expression vectors that provide for expression of nucleic acids of the invention in appropriate host cells.

Additionally provided are host cells comprising nucleic acids of the invention. Also provided are host cells comprising vectors of the invention. The invention also provides methods of producing enzymes encoded by nucleic acids of the invention, which methods comprise culturing host cells of the invention.

Also provided are isolated enzymes encoded by nucleic acids of the invention.

Detection of Impaired Alleles

The methods disclosed herein (e.g., methods of screening, preventing, and/or treating a condition or disease associated with impaired alleles of genes involved in metabolic pathways) generally require detecting the presence or absence of a plurality of single nucleotide polymorphisms (SNPs) in at least one enzyme-encoding gene within a metabolic pathway that may result in an impaired allele; preferably a plurality of known SNPs in the test gene. Alleles and/or predetermined sequence SNPs may be detected by allele specific hybridization, a sequence-dependent-based technique which permits discrimination between normal and impaired alleles. An allele specific assay is dependent on the differential ability of mismatched nucleotide sequences (e.g., normal:impaired) to hybridize with each other, as compared with matching (e.g., normal:normal or impaired:impaired) sequences.

A variety of methods are available for detecting the presence of one or more single nucleotide polymorphic in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods may require amplification of the test gene, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the alleles permitted to hybridize to a target molecule obtained from a particular animal or human. If the allele on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the allele of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of an allele. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the allele will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to an allele. The labeled terminator that is incorporated is thus determined by, and complementary to, the nucleotide present in the allele of the test gene. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying alleles in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at an allele. In such a format, since the signal is proportional to the number of deoxynucleotides incorporated, single nucleotide polymorphisms that occur in runs of the same nucleotide can result in signals that are proportional to the length of the run (Syvanen, A.-C., et al., Amer. J. Hum. Genet. 52:46-59 (1993)).

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express an enzyme-encoding gene.

Detection methods may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of an enzyme encoding gene.

Detection of Impaired Alleles Using Allele Specific Hybridization

A variety of methods well-known in the art can be used for detection of impaired alleles by allele specific hybridization. Preferably, the test allele is probed with allele specific oligonucleotides (ASOs); and each ASO comprises the sequence of a known allele. ASO analysis detects specific sequence substitutions in a target polynucleotide fragment by testing the ability of an allele specific oligonucleotide probe to hybridize to the target polynucleotide fragment. Preferably, the allele specific oligonucleotide probe contains the sequence (or its complement) of an impaired allele. The presence of an impaired allele in the target polynucleotide fragment is indicated by hybridization between the allele specific oligonucleotide probe and the target polynucleotide fragment under conditions in which an oligonucleotide probe containing the sequence of a wildtype allele does not hybridize to the target polynucleotide fragment. A lack of hybridization between the allele specific oligonucleotide probe having the sequence of the impaired allele and the target polynucleotide fragment indicates the absence of the impaired allele in the target fragment.

In one embodiment, the test gene(s) may be probed in a standard dot blot format. Each region within the test gene that contains the sequence corresponding to the ASO is individually applied to a solid surface, for example, as an individual dot on a membrane. Each individual region can be produced, for example, as a separate PCR amplification product using methods well-known in the art (see, for example, the experimental embodiment set forth in Mullis, K. B., 1987, U.S. Pat. No. 4,683,202).

Membrane-based formats that can be used as alternatives to the dot blot format for performing ASO analysis include, but are not limited to, reverse dot blot, (multiplex amplification assay), and multiplex allele-specific diagnostic assay (MASDA).

In a reverse dot blot format, oligonucleotide or polynucleotide probes, e.g., having known sequence are immobilized on the solid surface, and are subsequently hybridized with the sample comprising labeled test polynucleotide fragments. In this situation, the primers may be labeled or the NTPs maybe labeled prior to amplification to prepare a sample comprising labeled test polynucleotide fragments. Alternatively, the test polynucleotide fragments may be labeled subsequent to isolation and/or synthesis In a multiplex format, individual samples contain multiple target sequences within the test gene, instead of just a single target sequence. For example, multiple PCR products each containing at least one of the ASO target sequences are applied within the same sample dot. Multiple PCR products can be produced simultaneously in a single amplification reaction using the methods of Caskey et al., U.S. Pat. No. 5,582,989. The same blot, therefore, can be probed by each ASO whose corresponding sequence is represented in the sample dots.

A MASDA format expands the level of complexity of the multiplex format by using multiple ASOs to probe each blot (containing dots with multiple target sequences). This procedure is described in detail in U.S. Pat. No. 5,589,330 by A. P. Shuber, and in Michalowsky et al., American Journal of Human Genetics, 59(4): A272, poster 1573 (October 1996), each of which is incorporated herein by reference in its entirety. First, hybridization between the multiple ASO probe and immobilized sample is detected. This method relies on the prediction that the presence of a mutation among the multiple target sequences in a given dot is sufficiently rare that any positive hybridization signal results from a single ASO within the probe mixture hybridizing with the corresponding impaired allele. The hybridizing ASO is then identified by isolating it from the site of hybridization and determining its nucleotide sequence.

Suitable materials that can be used in the dot blot, reverse dot blot, multiplex, and MASDA formats are well-known in the art and include, but are not limited to nylon and nitrocellulose membranes.

When the target sequences are produced by PCR amplification, the starting material can be chromosomal DNA in which case the DNA is directly amplified. Alternatively, the starting material can be mRNA, in which case the mRNA is first reversed transcribed into cDNA and then amplified according to the well known technique of RT-PCR (see, for example, U.S. Pat. No. 5,561,058 by Gelfand et al.).

The methods described above are suitable for moderate screening of a limited number of sequence variations (e.g., impaired alleles). However, with the need in molecular diagnosis for rapid, cost effective large scale screening, technologies have developed that integrate the basic concept of ASO, but far exceed the capacity for mutation detection and sample number. These alternative methods to the ones described above include, but are not limited to, large scale chip array sequence-based techniques. The use of large scale arrays allows for the rapid analysis of many sequence variants. A review of the differences in the application and development of chip arrays is covered by Southern, E. M., Trends In Genetics, 12:110-115 (March 1996) and Cheng et al., Molecular Diagnosis, 1:183-200 (September 1996). Several approaches exist involving the manufacture of chip arrays. Differences include, but not restricted to: type of solid support to attach the immobilized oligonucleotides, labeling techniques for identification of variants and changes in the sequence-based techniques of the target polynucleotide to the probe.

A promising methodology for large scale analysis on ‘DNA chips’ is described in detail in Hacia et al., Nature Genetics, 14:441-447 (1996), which is hereby incorporated by reference in its entirety. As described in Hacia et al., high density arrays of over 96,000 oligonucleotides, each 20 nucleotides in length, are immobilized to a single glass or silicon chip using light directed chemical synthesis. Contingent on the number and design of the allele specific oligonucleotide probe, potentially every base in a sequence can be interrogated for alterations. Allele specific oligonucleotide probes applied to the chip, therefore, can contain sequence variations, e.g., SNPs, that are not yet known to occur in the population, or they can be limited to SNPs that are known to occur in the population.

Prior to hybridization with allele specific olignucleotide probes on the chip, the test sample is isolated, amplified and labeled (e.g. fluorescent markers) by means well known to those skilled in the art. The test polynucleotide sample is then hybridized to the immobilized allele specific oligonucleotide probes. The intensity of sequence-based techniques of the target polynucleotide fragment to the immobilized allele specific oligonucleotide probe is quantitated and compared to a reference sequence. The resulting genetic information can be used in molecular diagnosis. A common, but not limiting, utility of the ‘DNA chip’ in molecular diagnosis is screening for known SNPs. However, this may impose a limitation to the technique by only looking at mutations that have been described in the field. The present invention allows allele specific hybridization analysis be performed with a far greater number of mutations than previously available. Thus, the efficiency and comprehensiveness of large scale ASO analysis will be broadened, reducing the need for cumbersome end-to-end sequence analysis, not only with known mutations but in a comprehensive manner all mutations which might occur as predicted by the principles accepted, and the cost and time associated with these cumbersome tests will be decreased.

Accordingly, in one aspect, the invention provides methods for detecting impaired alleles of enzyme-encoding genes or enzyme-encoding nucleic acids. For example, provided herein are methods for detecting alleles of MTHFR, ATIC, CBS, CTH, GART, MAT1A, MAT2A, and MTHFS.

In one embodiment, detecting an SNP in an enzyme-encoding nucleic acid involves nucleic acid sequencing. In one embodiment, detecting a mutation in an enzyme-encoding nucleic acid involves PCR. In one embodiment, detecting a mutation in an enzyme-encoding nucleic acid involves RFLP analysis. In one embodiment, detecting a mutation in an enzyme-encoding nucleic acid involves nucleic acid hybridization. Detecting amutation SNP through hybridization may be done, for example, using a nucleic acid array comprising a nucleic acid that will hybridize under stringent conditions to an enzyme-encoding nucleic acid, or a fragment thereof, comprising such an SNP.

In one embodiment, the methods comprise use of an in vivo assay for determining the activity of an allele of an enzyme-encoding gene, as described herein.

Combinations of methods may also be used to detect and characterize an impaired allele of an enzyme-encoding gene. In one embodiment, the methods comprise use of an in vivo assay for determining theactivity of an enzyme-encoding gene, as described herein, and detecting an SNP in an enzyme-encoding nucleic acid.

In one embodiment, the methods comprise use of an in vivo assay for determining enzyme activity, as described herein, and a temperature sensitivity assay to determine enzyme stability at an elevated temperature.

In one embodiment, the methods comprise use of an in vivo assay for determining enzyme activity, as described herein, and an in vitro assay for determining the specific activity of the enzyme.

In a preferred embodiment, an impaired allele of MTHFR comprises a non-synonymous substitution that encodes for a mutation in the MTHFR protein selected from the group consisting of M110I, H213R, D223N, D291N, R519C, R519L, and Q648P. In an especially preferred embodiment, an impaired allele comprises a non-synonymous substitution that encodes for a mutation in the MTHFR protein selected from the group consisting of M110I, H213R, D223N, and D291N.

### Yeast Strains

In one aspect, the invention provides yeast strains capable of detecting impaired alleles of enzymes involved in folate/homocysteine metabolism. Such yeast strains are useful in methods disclosed herein. The yeast strains comprise a first mutation allowing for complementation by a functionally homologous enzyme of interest, and a second mutation (or group of mutations) rendering the strain dependent upon supplementation with a cofactor for an assayable phenotype related to function of the first gene.

In one embodiment, the invention provides yeast strains capable of detecting impaired alleles of CTH and determining the responsiveness thereof to vitamin B₆. In a preferred embodiment, the yeast strain comprises a mutation in cys3 and in sextuple-delete sno1Δ sno2Δ sno3Δ snz1Δ snz2Δ snz3Δ.

In one embodiment, the invention provides yeast strains capable of detecting impaired alleles of CBS and determining the responsiveness thereof to vitamin B₆. In a preferred embodiment, the yeast strain comprises a mutation in cys4 and in sextuple-delete sno1Δ sno2Δ sno3Δ snz1Δ snz2Δ snz3Δ.

In one embodiment, the invention provides yeast strains capable of detecting impaired alleles of MTHFR and determining the responsiveness thereof to folate. In a preferred embodiment, the yeast strain comprises a mutation in met13 and fol3.

Screening for Risk of Disease

In one aspect, the invention provides methods of screening for risk of a condition or disease associated with aberrant folate/homocysteine metabolism. The methods involve screening for an impaired allele of a gene involved in folate/homocysteine metabolism, as described herein.

In one embodiment, the invention provides methods of screening for a risk of a disease or condition associated with an enzyme dysfunction, wherein the enzyme is selected from the group consisting of MTHFR, ATIC, MTHFS, MAT1A, MAT2A, and GART. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birthweight, recurrent early spontaneous abortion, thrombosis, retinal artery occlusion, down's syndrome, colorectal cancer, breast cancer, lung cancer, prostate cancer, depression, schizophrenia, Alzheimers disease/dementia, age-related macular degeneration, and glaucoma. The methods comprise use of a method for detecting an impaired allele selected from the group consisting of an impaired allele of MTHFR, an impaired allele of ATIC, an impaired allele of MTHFS, an impaired allele of MAT1A, an impaired allele of MAT2A, and an impaired allele of GART, as described herein.

In one embodiment, the invention provides methods of screening for a risk of a disease or condition associated with CBS dysfunction. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birthweight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma. The methods comprise use of a method for detecting an impaired CBS allele, as described herein.

In one embodiment, the invention provides methods of screening for a risk of a disease or condition associated with CTH dysfunction. In a preferred embodiment, the disease or condition is selected from the group consisting of cardiovascular disease, coronary artery disease, ischemic stroke, atherosclerosis, neural tube defects, orofacial clefts, pre-eclampsia, pre-term delivery/low birthweight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma. The methods comprise use of a method for detecting an impaired CTH allele, as described herein.

Screening for Chemotherapeutic Response Potential

In one aspect, the invention provides methods of determining an individual's chemotherapeutic response potential. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in an individual indicates a decreased response potential.

In a preferred embodiment, the chemotherapeutic is methotrexate or 5-fluorouracil.

Screening for Chemotherapeutic Toxicity

In one aspect, the invention provides methods of determining chemotherapeutic toxicity for an individual. The methods comprise use of a method for detecting an impaired allele of a gene involved in folate/homocysteine metabolism, as described herein. In a preferred embodiment, the gene is selected from the group consisting of MTHFR, ATIC, MTHFS, MAT1A, MAT2A, and GART. Detection of an impaired allele in an individual indicates an increased toxicity potential.

In a preferred embodiment, the chemotherapeutic is methotrexate or 5-fluorouracil.

Prophylaxis and Treatment

In one aspect, the invention provides methods of preventing a condition or disease associated with metabolic enzyme deficiency. The methods comprise increasing an individual's intake of an cofactor based on information obtained from the foregoing assays and methods, which inform on the presence of cofactor-sensitive impaired alleles. In a preferred embodiment, the methods comprise detecting a cofactor-remediable impaired allele of a metabolic gene, as described herein.

In one embodiment, the invention provides methods of preventing a condition or disease associated with aberrant folate/homocysteine metabolism. The methods comprise increasing an individual's intake of folate and/or vitamin B₆. In a preferred embodiment, the methods comprise detecting an impaired allele of a gene involved in folate/homocysteine metabolism, as described herein.

In one embodiment, the invention provides a method of preventing a condition or disease associated enzyme dysfunction in an individual having an impaired allele of an enzyme-encoding gene that is cofactor remediable, wherein the enzyme-encoding gene is selected from the group consisting of MTHFR, ATIC, MTHFS, MAT1A, MAT2A, and GART. The method comprises increasing the individual's intake of folate.

In one embodiment, the invention provides a method of preventing a condition or disease associated CBS dysfunction in an individual having an impaired CBS allele. The method comprises increasing the individual's intake of vitamin B₆.

In one embodiment, the invention provides a method of preventing a condition or disease associated CTH dysfunction in an individual having an impaired CTH allele. The method comprises increasing the individual's intake of vitamin B₆.

In one aspect, the invention provides methods of treating a condition or disease associated with aberrant folate/homocysteine metabolism. The methods comprise increasing an individual's intake of folate and/or vitamin B₆. In a preferred embodiment, the methods comprise detecting an impaired allele of a gene involved in folate/homocysteine metabolism, as described herein.

In one embodiment, the invention provides a method of treating a condition or disease associated with enzyme dysfunction in an individual having an impaired allele of an enzyme-encoding gene that is co-factor remediable, wherein the enzyme-encoding gene is selected from the group consisting of MTHFR, ATIC, MTHFS, MAT1A, MAT2A, and GART remediable by cofactor, wherein the. The method comprises increasing the individual's intake of folate.

In one embodiment, the invention provides a method of treating a condition or disease associated CBS dysfunction in an individual having an impaired CBS allele. The method comprises increasing the individual's intake of vitamin B₆.

In one embodiment, the invention provides a method of treating a condition or disease associated CTH dysfunction in an individual having an impaired CTH allele. The method comprises increasing the individual's intake of vitamin B₆.

### EXAMPLES

### Example 1

### Prevalence of Folate-Remedial MTHFR Enzyme Variants in Humans

The prevalence of folate-remediable MTHFR enzyme variants from a large population to determine the incidence and impact of low frequency variation and explore the phenomenon of vitamin remediation. From over 500 individuals, 14 different non-synonymous substitutions were identified, 5 of which impaired enzyme function. While all deleterious alleles were at least somewhat folate responsive, 4 of the 5 mutant proteins could be fully restored to normal levels by elevating intracellular folate levels.

### Example 1.1

### Methods

DNA Sample Population. DNA samples were from the Coriell Institute Cell Repository (Camden, N.J., USA).

MTHFR Exon Sequencing. 11 MTHFR coding exons were sequenced in the above samples by PCR sequencing using primer pairs commercially available from the Variant SeqR product line (Applied Biosystems, Foster City, Calif.) and according to the protocols supplied. The exon regions sequenced corresponded to NCBI MTHFR reference sequences for mRNA (NM_{—}005957) and the corresponding protein (NP_{—}005958) of 656 amino acids. Sequencing amplicon and probe information is available at http://www.ncbi.nim.nih.gov/qenome/probe for the following target amplicons:

Exon 1 (RSA000045684); Exon 2 (RSA000045680); Exon 3 (RSA000577249); Exon 4 (RSA000045678); Exon 5 (RSA000045676); Exon 6 (RSA001308795); Exon 7 (RSA001253193); Exon 8 (RSA000045669); Exon 9 (RSA000580767); Exon 10 (RSA 000580766); Exon 11 (RSA000580765, RSA000027240). Only the portion of exon 11 that spanned the coding region was sequenced. To ensure high confidence in base-calling, only high-quality reads were used for analysis (average QV scores >40 for the region that spanned the target exon; all exons were covered by double-strand reads). Based on these filtering criteria, success rates ranged from 89.9% to 95% for each exon (see Table I). All sequence information was analyzed using the SeqScape software suite (Applied Biosystems). 3 As a quality control measure, a subset of base calls were directly verified by TaqMan (Applied Biosystems) allelic-discrimination assays and compared with publicly available genotype data as described below.

Plasmids. Plasmid phMTHFR, which carries the 5′-end HA (hemagglutinin A) epitope-tagged human MTHFR open reading frame (reference protein sequence NP_{—}005948) under the control of the inducible yeast GAL1 promoter and the URA3 selectable marker, was a generous gift of Warren Kruger (Shan et al., 1999, supra). This plasmid served as the backbone to reconstruct all MTHFR variants by site-directed mutagenesis using the QuikChange kit (Stratagene). Integrating plasmids containing galactose-inducible MTHFR variants were created by PCR cloning the fragment containing URA3, the GAL1 promoter and MTHFR coding region from the phMTHFR-based plasmid into pHO-poly-HO (Voth et al., 2001, Nucleic Acids Res. 29:e59), which enables targeted integration of this cassette at the HO locus.

Strains. All haploid yeast strains were MATa his3 leu2 ura3 lys2 in the S288c background (Brachman et al., 1998, Yest 14:115-32). MATa/MATα diploid strains were created by mating isogenic MATa and MATα strains. fol3Δ::KanMX and fol3Δ::KanMX met13Δ::KanMX strains were obtained by standard mating/sporulation techniques using strains from the S. cerevisiae gene-knockout collection (invitrogen). Diploids (homozygous or heterozygous for MTHFR variants) were created by mating fol3Δ::KanMX met13Δ::KanMX haploids that each contain an integrated version of the GAL 1:MTHFR variant cassette.

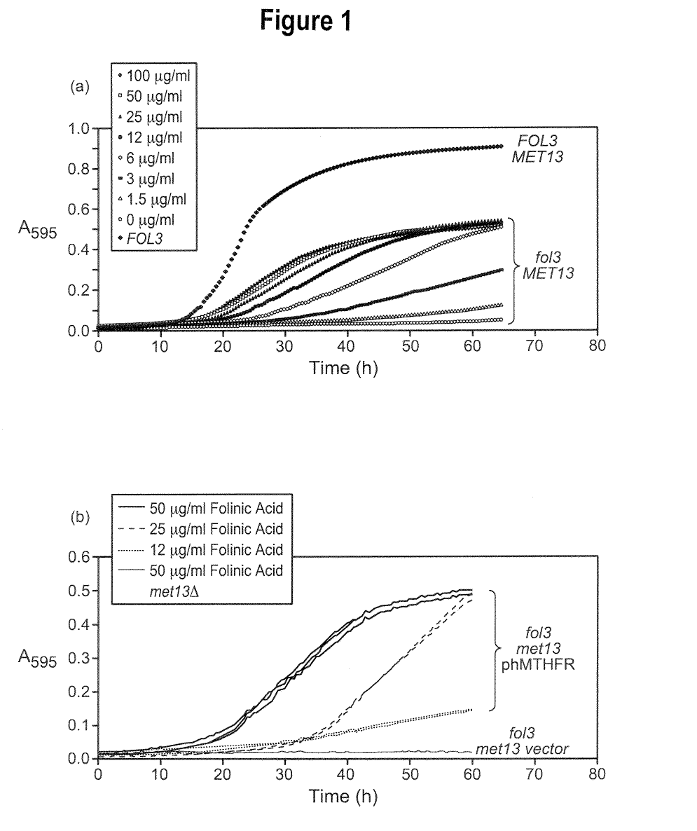

Growth Conditions. Synthetic growth media lacking folate was minimal media (Sherman, 2002, Genetics & Molecular Biol., eds. Guthrie and Fink (Academic, New York), pp. 3-41) with Yeast Nitrogen Base without Vitamins (Obiogene), and all vitamins except folate added back individually. All fol3Δ::KanMX cells were supplemented with 50 μg/ml folinic acid (Sigma). For kinetic growth measurements, fol3Δ::KanMX met13Δ::KanMX cells were transformed with GAL1 promoter-driven MTHFR variants and grown to log phase in synthetic galactose medium (2% galactose, 0.1% glucose) supplemented with folinic acid (50 μg/ml) and methionine (20 μg/ml). Cells were washed 3 times and aliquoted into 96-well plates containing fresh galactose media with varying amounts of folinic acid, but lacking methionine. The volume per well was 200 μl with a starting cell density of OD₅₉₅=0.01. Absorbance was tracked every 15-30 minutes for at least 60 hours in a Tecan GENios plate reader at 30° C. with no shaking. MET13 cells used in FIG. 1a were treated the same way except that all growth was in the absence of methionine.

MTHFR enzyme activity assay. The assay, which measures the reverse reaction of that catalyzed by MTHFR under physiological conditions, was as described (Shan et al., 1999, supra) with the following modifications: Yeast extracts were created by bead lysis of 40 OD₅₉₅ cell equivalents (fol3Δ met13Δ cells supplemented with folinic acid and methionine as above) in 350 μl of Lysis Buffer (100 mM Sucrose, 50 mM KHPO₄ (pH 6.3), protease inhibitor cocktail). Extracts were clarified by a brief microcentrifugation, and 10-200 μg of extract used to determine the linear range of activity. Radiolabeled substrate (5-[¹⁴C]MeTHF) was from GE Healthcare Life Sciences. For heat treatment, the reaction mixes without 5-[¹⁴C]MeTHF were heated to 55° C. for the indicated times at which point 5-[¹⁴C]MeTHF was added back and the reaction proceeded.

MTHFR Immunoblot analysis. 10 OD₅₉₅ cell equivalents (fol3Δ met13Δ cells supplemented with folinic acid and methionine as above) were extracted in 200 μl 0.1 M NaOH for 15 min. 50 μl SDS sample buffer (0.5M Tris 6.8, 0.4% SDS) was added to supernatants, which were then boiled, clarified and subject to SDS-PAGE. HA-tagged MTHFR variants were detected on a LI-COR Infrared Imager. Mouse monoclonal anti-HA antibody was from Sigma. Yeast 3-Phosphoglycerate kinase (Pgkl p), a loading control, was detected by mouse antibodies generously donated by Jeremy Thorner (University of California, Berkeley, Calif.).

### Example 1.2

### Results

MTHFR variants in humans. The entire coding region of human MTHFR was sequenced by amplifying the coding portion in each of 11 exons from 564 individuals of diverse ethnicities. The lengths of the coding regions, the number of alleles interrogated and all nonsynonymous substitutions are listed in Table 3. In all, 2,081,106 bp of coding DNA, and sampled every exon to a depth of over 1,000 alleles were analyzed. These data revealed 14 nonsynonymous changes, 11 of which show a minor allele frequency (MAF) <1%, with 7 alleles seen only once. Some low-frequency alleles were seen previously (see Table 3). The number of low-frequency nonsynonymous substitutions was in good agreement with other studies that sampled deeply into random populations (Martin et al., 2006, Pharmacogenet Genomics 16:265-77; Livingston, 2004, Genome Res 14:1821-31; Glatt et al., 2001, Nat. Genet. 27:435-38). In addition, 3 well-studied common substitutions were observed that displayed the expected global population frequencies (A222V-29.3%, E429A-23.6%, R594Q-4.4%).

As a quality-control check on the accuracy of the base-calling, 8 variants (including 4 singletons) were reanalyzed by TaqMan alielic-discrimination assays in 100 samples that were independently PCR-amplified and saw 100% concordance of the data. Furthermore, population genotyping data from the Environmental Genome Project (http://www.niehs.nih.gov/envgenomI) and Perlegen (Mountain View, Calif.), which both used Coriell samples that overlap some in this study (dbSNP build 127) were in concordance in 814 of 817 (99.6%) genotype calls. For two of the three discordant loci, our sequence data were unambiguous and appeared correct.

Complete coding region sequences were obtained for 480 individuals. Eighteen (4%) were carriers of a low-frequency nonsynonymous variant. Significantly, the combination of the 3 common polymorphisms (A222V, E429A and R594Q) with the range of the low frequency changes led to a great deal of individual heterogeneity. Twenty-eight different nonsynonymous genotypes were observed in this group whose haplotype, in most cases, could not be deduced from the data.

MTHFR-folate interaction in vivo. Because the clinical significance of genetic variants lies in their functional consequence, all nonsynonymous changes were tested for their effect on MTHFR function, and importantly, whether or not impaired alleles displayed folate-responsiveness. Folate auxotrophy (fol3) was introduced into a met13 strain, allowing titration of intracellular folate concentrations by varying folinic acid in the growth media. Folinic acid (5-formyl-tetrahydrofolate) can be metabolized in yeast to methenyl-tetrahydrofolate, which in turn can be converted to other folate coenzymes (Cherest et al. (2000) J. Biol. Chem. 275:14056-63). In this way, human MTHFR functionality (growth in the absence of methionine) was measured as a function of increasingly limiting cellular folate status.

Under these conditions, folinic acid supplementation above 50 μg/ml did not confer any significant growth advantage (FIG. 1a). However, at concentrations below 50 μg/ml, growth clearly correlated with available folinic acid in the medium. Thus intracellular folate levels were rate-limiting in this range. When compared to growth of FOL3 cells, folinic acid supplementation did not completely compensate for lack of endogenous folate biosynthesis. However, this gap was mostly reflected in the density at which cells entered stationary phase rather than growth rate, perhaps reflecting limitations in folinic acid uptake, or in the utilization of folinic acid as the sole folate source.

The ability of human MTHFR to complement fol3 met13 cells was a function of folinic acid supplementation in the media (FIG. 1b). As for folate supplementation, expression of human MTHFR from the GALL promoter did not completely compensate for loss of Met13p (compare FIG. 1b with fol3 MET13 cells at equivalent folate doses in FIG. 1a). Thus, below 50 μg/ml folinic acid, both folate and MTHFR were rate-limiting for growth, allowing even subtle changes in MTHFR activity to be reflected in the growth readout. Note that folinic acid supplementation above 50 p g/ml did not confer a significant growth advantage to cells expressing either the endogenous yeast MTHFR (MET13; FIG. 1a) or the major human allele (FIG. 1b), but was beneficial for impaired alleles of MTHFR (see below).

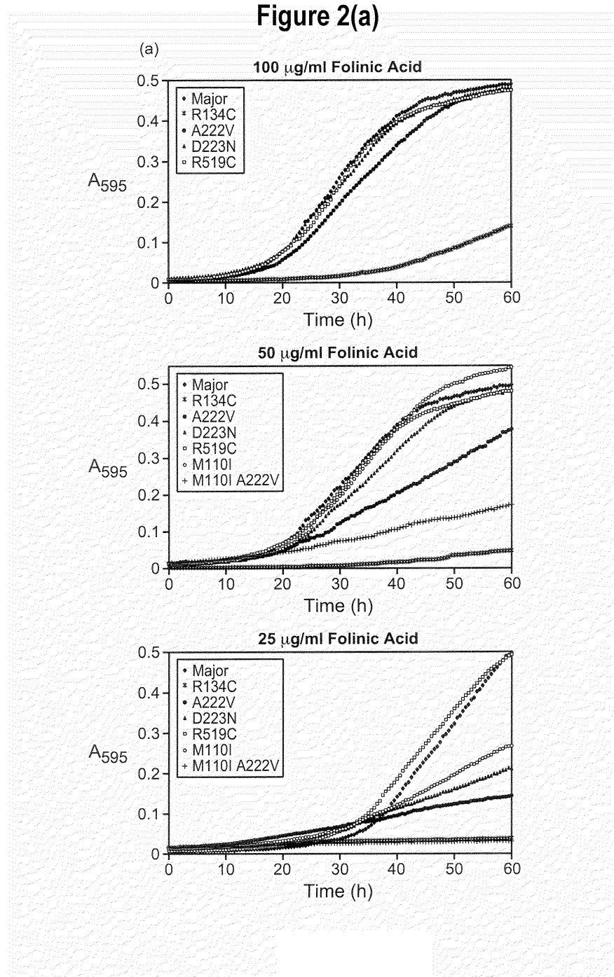

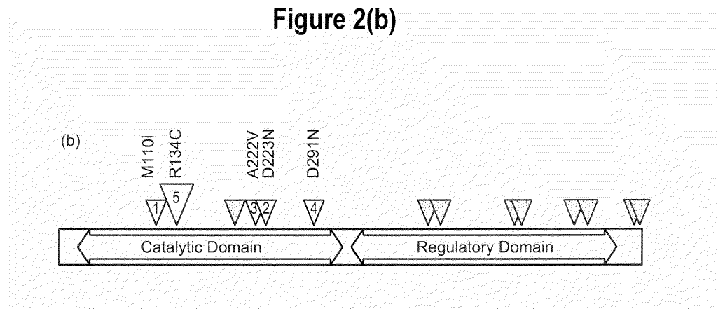

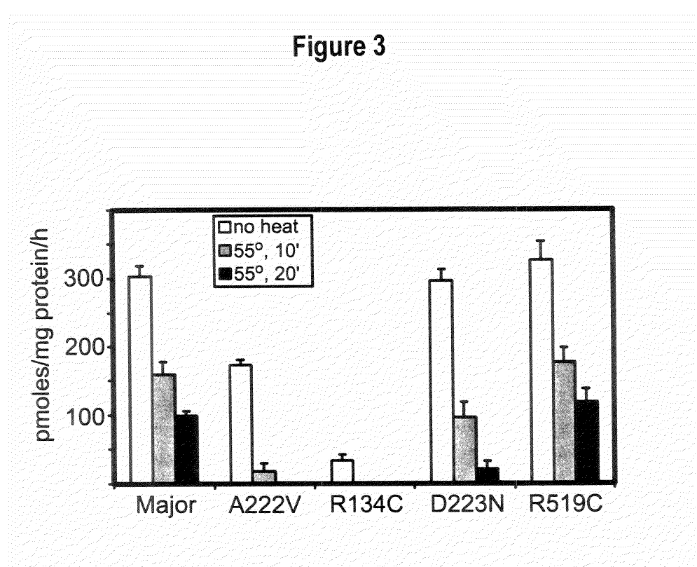

Functional impact of MTHFR variants. Five nonsynonymous alleles tested over a range of folate concentrations illustrated the range of functional effects observed (FIG. 2a). There was nearly complete restoration of function of the A222V variant at 100 p g/ml folinic acid and significantly less activity (relative to the major allele) at a four-fold lower level of supplementation (25 μg/ml). Thus, under these conditions the known folate remediability of the A222V defect was recapitulated. The exact intracellular concentrations of reduced folates in yeast under these conditions was unknown. Nevertheless, the behavior of the A222V allele effectively calibrated the intracellular concentrations in yeast and human cells. The A222V enzyme has approximately 50% the intrinsic activity of common allele (Martin, 2006, Pharmacogenet. Genomics 16:265-77; Rozen, 1997, Thromb. Haemost. 78:523-26) and 50% reduction in growth rate was observed at 50 p g/ml folate supplementation. Furthermore, the same 50% drop in A222V enzyme activity in cell-free assays from cells grown at 50 μg/ml folinic acid was observed (FIG. 3, below). Thus, the behavior of A222V in yeast recapitulated its behavior in human cells.

Four low-frequency alleles were tested in the same way (FIG. 2a). R519C appeared benign since growth was unaffected at all folate concentrations. R134C was severely impaired at all folate concentrations, though activity was somewhat folate-responsive. The D223N and M1101 alleles displayed folate-remedial activity similar to A22V (though less severely impaired) in that growth was similar to the major allele at, or above, 50 μg/ml folinic acid, but functioned poorly below 50 μg/ml folinic acid.

The MTHFR enzyme has an N-terminal catalytic domain and a C-terminal regulatory domain, which binds the allosteric inhibitor S-adenosylmethionine (AdoMet; Sumner et al., 1986, J. Biol. Chem. 261:7697-7700). Of the 6 alileles that fell within the catalytic domain (M110I, R134C, H213R, A222V, D223N and D291N), only H213R was benign (FIG. 2b). M110I, A222V, D223N and D291N displayed folate-remedial behavior in that these enzyme variants were similar to the major allele at higher concentrations of folate supplementation (50-200 μg/ml folinic acid), but were considerably weakened as folate became more rate-limiting. The R134C variant never approached the capacity of the major allele to support growth at any level of folate supplementation and hence was classified as a responsive, but not a remedial allele. All substitutions within the regulatory domain (from G422R through T653M) behaved similarly to the major allele (FIG. 2b).

Synergistic interactions between amino acid substitutions. The distribution of variants implied the existence of compound alleles containing two (or more) substitutions. Therefore several compound alleles (based upon their occurrence in individual samples) were created to test whether allele combinations lead to synergistic or suppressive effects. For A222V combinations with common variants (A222V E429A and A222V R594Q), minor allele homozygotes were observed for at least one of the alileles and therefore are sure that such variants exist. However, for the low frequency variants, both the A222V variant and the novel variants always occurred as heterozygotes. Since the haplotype is unknown, these individuals could harbor either the two single substitution alieles or a compound allele. Therefore all possible double-substitution alileles were created and tested their function (e.g. M1101 A222V, FIG. 2a). At the two folinic acid concentrations tested, the M1101 A222V variant functioned more poorly than the sum of the individual alleles, indicating synergistic defects in compound alleles. At 50 μg/ml folinic acid, the M1101 variant was nearly indistinguishable from the major allele, yet it significantly enhanced the A222V defect. For all combinations tested, alleles that affected function individually (M1101 and D291N) synergized when combined with A222V, whereas benign changes did not enhance the A222V defect.

Biochemical assays recapitulated in vivo function. To evaluate the reliability of the growth assay, cell-free MTHFR enzyme assays were performed for all variants in crude yeast lysates (see Materials and Methods). In addition to measuring specific activity, variants were tested for thermolability (a measure of enzyme stability) by heat treatment at 55° C. for various times. There was a good correlation between intrinsic activity and growth rate (FIG. 3; compare the activities of non heat-treated samples for the major MTHFR allele, A222V and R134C with the growth curves in FIG. 2). Again, the A222V variant displayed approximately 50% of the enzymatic activity of the major allele, as reported previously (20, 25, 34). As in the growth assay, the R519C variant exhibited similar activity to the major allele and was representative of all changes in the regulatory domain including the common E429A variant (data not shown). Although there have been reports that E429A affects enzyme function (27), our data agreed with others (10, 20, 25) that this change was benign.

The A222V mutant enzyme is less stable and more thermolabile than the major form (Guenther et al., 1999, Nat. Struct. Biol. 6:359-65; Yamada et al., 2001, Proc. Natl. Acad. Sci. 98:14853-58) and folate remediation of this variant is thought to occur by promoting stabilization of the protein. Under the conditions used here (55° C., 20 m), A222V lost nearly all activity while the major allele retained about 30% of its original activity, in agreement with previous studies (20). The novel D223N allele also displayed increased thermolability that may similarly explain folate-remediability in this case, although the enzyme defect was not as great.

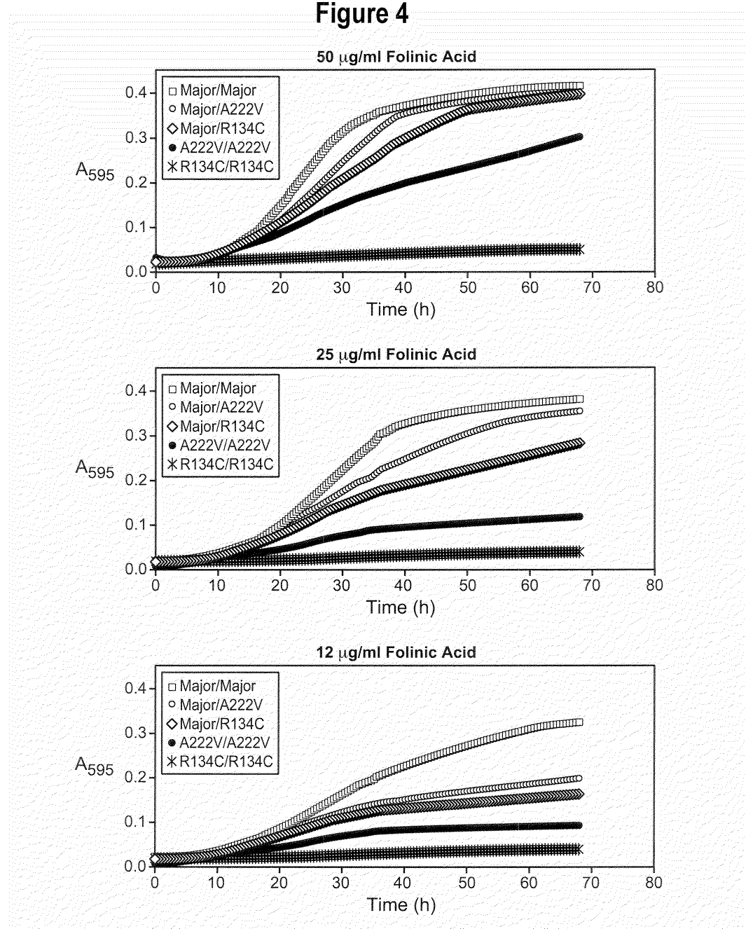

Heterozygote phenotypes. Since low frequency alleles usually occur as heterozygotes, their significance tends to be dismissed. To understand better the functional significance of heterozygosity of MTHFR alleles, diploid yeast with two copies of human MTHFR were created by mating haploid strains that each have either the same allele expressed from an integrated expression cassette (homozygotes) or different alleles to create heterozygotes (see methods). As above, these strains were tested for growth as a function of folate supplementation (FIG. 4). Heterozygotes displayed a growth phenotype in this assay that was exacerbated under conditions of limiting folate, indicating that the reduced-function alleles were codominant with wild type.

Cellular MTHFR activity as measured in the growth assay appeared to reflect additive effects of alleles. Furthermore, additional experiments with hemizygotes (diploids with a single integrated expressed allele; data not shown) demonstrated that the formation of heterodimers between major and minor alleles in heterozyotes offered little or no rescue of mutant alleles. For example, diploid MTHFR major allele/null cells (hemizygotes) behaved similarly to major allele/R134C heterozygotes under all conditions, and similarly to major allele/A222V heterozygotes in low folate media (where A222V is inactivated). Thus, the phenotypic contribution of deleterious alleles in heterozygote cells was easily observed, raising the possibility of more widespread phenotypic consequences from heterozygosity in the human genome.

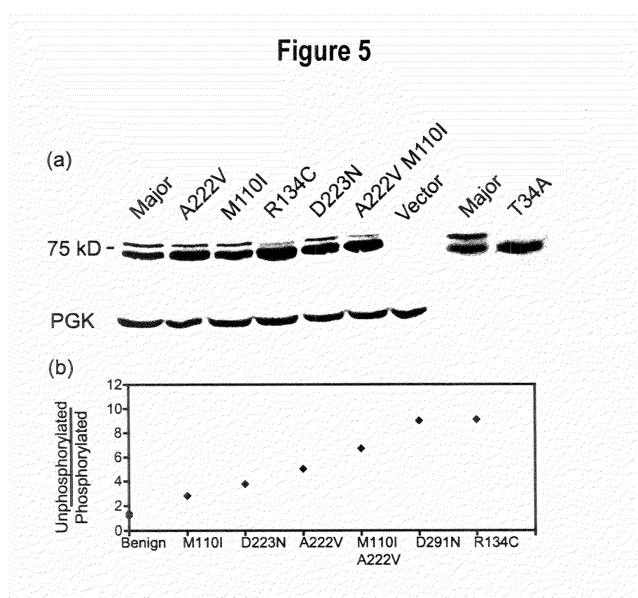

Modification of MTHFR variants in yeast by phosphorylation. The abundance of MTHFR variant proteins was determined by immunoblotting using antibodies directed against the N-terminal hemagglutinin A (HA) epitope tag (FIG. 5a). In all samples, the protein ran as a doublet of approximately 72 kD and 78 kD. This pattern closely resembled that observed for human MTHFR expressed in insect cells (37), where the upper band represents MTHFR multiply-phosphorylated near the N-terminus. Phosphorylation of MTHFR in insect cells is dependent on a threonine residue at position 34 and substitution of this threonine to alanine (T34A) results in an enzyme that is unable to be phosphorylated (37). This mutation had the same effect on human MTHFR expressed in S. cerevisiae and indicated that, as in insect cells, the upper band was phosphorylated MTHFR (FIG. 5a).

The role of phosphorylation of MTHFR is suggested to be involved in negative regulation (37). In support of this hypothesis, the phosphorylation pattern observed here directly correlated with cellular MTHFR activity. Specifically, the ratio of the abundance of the unphosphorylated:phosphorylated forms increased with decreasing activity (FIG. 5b). Interestingly, the overall abundance of all variants (phosphorylated plus unphosphorylated forms) did not appear to be strikingly different. This might not be expected if deleterious substitutions affected intrinsic enzyme stability, unless other factors are involved in determining protein levels.

All functionally impaired alleles clustered in the N-terminal, catalytic half of MTHFR (36) which contains the folate and FAD binding sites. On the other hand, 8 nonsynonymous substitutions in the C-terminal regulatory domain of MTHFR were identified and all 8 appeared benign in both the complementation and cell-free enzyme assays. Furthermore, no synergy was seen between regulatory domain substitutions and A222V in compound alleles (FIG. 2). Either these alterations were neutral, as has been reported for E429A (10, 20, 25), or the assay was insensitive to their defect, This finding however was consistent with the observation that most mutations in MTHFR that result in severe clinical phenotypes occur in the catalytic domain (http://www.hqmd.cf.ac.uk/ac/index.php). The regulatory domain has been proposed to play a role in stabilization of the catalytic domain (20). If so, this role may be somewhat tolerant to amino acid substitutions and may explain how a chimeric MTHFR composed of the S. cerevisiae N-terminal domain fused to the Arabidopsis C-terminal domain (equivalent to approximately 50 nonsynonymous substitutions of the yeast enzyme in the regulatory domain) does not harm enzyme activity (38). It should be noted that Martin et al (25) reported that the common R594Q variant in the C-terminal domain affected enzyme activity when expressed in COS-1 cells. This change appeared benign, however, in cell-based and cell-free assays of the enzyme expressed in yeast. Although the reason for this discrepancy is unclear, it may be reflective of the host expression system since these authors observed only a single species of MTHFR (unknown phosphorylation status) in their immunoblot analyses.

The phenotypes of heterozygotes. The behavior of diploid yeast heterozygous for functionally impaired MTHFR alleles demonstrated that heterozygote phenotypes were clearly observable, especially under conditions of limiting folate (FIG. 4). The appearance of phenotypes in heterozygotes was significant since most genetic variation occurs as heterozygosity and low frequency alleles exist primarily as heterozygotes in the population. This result is consistent with the observations that cellular MTHFR activity in lymphocyte extracts is directly correlated with genotype: individuals heterozygous for A222V (A/V) have approximately 65% of the total activity seen for major allele (A/A) homozygotes, where A222V homozygotes (VN) retain 30% of the activity of A/A homozygotes (7). In a recent study examining the full spectrum of alleles in the adipokine ANGPTL4, which affects serum triglyceride levels, heterozygosity for the nonsynonymous E40K allele was significantly associated with lower plasma triglyceride levels (18). Thus, cases in which heterozygosity is phenotypically detectable increases the significance of the contribution of low frequency variants since there can be orders of magnitude more carriers than homozygotes. Note that heterozygote phenotypes was observed under conditions in which MTHFR activity was rate-limiting for cell growth. Whether or not enzymatic steps are rate-limiting in a particular pathway in humans depends on both genetic and environmental factors.

Mutations and MTHFH phosphorylation and abundance. Folate remediation of nonsynonymous changes in the catalytic domain may occur by protein stabilization (as for A222V; 9, 10) or by overcoming other aspects of molecular function such as cofactor Kₘ (2, 5). At least one deleterious allele, D223N, showed increased thermolability (FIG. 3) analogous to A222V, which argued for a stability defect. The hypothesis that folate-remedial alleles of MTHFR are those in which a folate species stabilizes unstable forms of the enzyme would suggest that the level of MTHFR protein be proportional to intrinsic activity of the variants, as has been suggested (25). However, our observations indicated that while phosphorylation status correlated with enzyme activity (FIG. 5), the overall abundance (phosphorylated plus unphosphorylated forms) did not appear to change strikingly (within a two-fold range). It is unlikely that phosphorylated MTHFR is the active form of the enzyme since Yamada et al (37) demonstrated an inhibitory effect of phosphorylation on intrinsic activity. Consistent with this, the behavior of the non-phosphorylatable T34A variant in both the growth and enzyme assays was similar to that of the major allele (data not shown). Furthermore, while low intracellular folate levels decrease MTHFR stability (as measured by abundance), this effect is not enhanced in variants that impair function. Because these results are at variance with the expected protein destabilization of deleterious changes, it was deduced there must be a compensatory regulatory response that is currently under investigation. In this way the activity of variants could be strikingly different (FIG. 2), whereas the overall protein abundance may not be (FIG. 5). While our results are consistent with feedback regulation by phosphorylation (37), the role of phosphorylation in turnover is unknown. In this vein, it will be interesting to determine the effect of the T34A change in combination with other impaired alleles.

The Folate/Homocysteine Metabolic Pathway

The Kyoto Encyclopedia of Genes and Genomes (KEGG) reference pathways database (www.genome.jp/kegg) depicts steps in folate and homocysteine metabolism. The pathways are linked via the Methionine Synthase reaction, and marginal folate deficiencies in cell cultures, animal model systems and in humans impair homocysteine remethylation (see, for example, Stover PJ. 2004. Physiology of folate and vitamin B12 in health and disease. Nutr Rev 62:S3-12). Homocysteine is a hypothesized risk factor for NTDs (see, for example, Mills et. al., 1995. Homocysteine metabolism in pregnancies complicated by neural tube defects. Lancet 345:149-1151). Folate deficiency also impairs methylation mediated by S-adenosyl-methionine (SAM; see, for example, Stover, supra), which is an allosteric inhibitor of both MTHFR and CBS (see, for example, Kraus et al., 1999. Cystathionine-o-synthase mutations in homocystinuria. Hum Mut 13:362-375; Daubner et al., 1982. In Flavins and Flavoproteins, eds. Massey, V. & Williams, C. H. (Elsevier, N.Y.), pp. 165-172). Furthermore, elevations in the S-adenosyl-homocysteine:S-adenosyl-methionine (SAH/SAM) ratios have been proposed in the mechanism of NTD development (see, for example, Stover, supra; Scott, 2001. Evidence of folic acid and folate in the prevention of neural tube defects. BibI Nutr Dieta 55:192-195. van der Put et al., 2001. Folate, Homocysteine and Neural Tube Defects: An Overview. Exptl Biol Med 226: 243-270.1, 5, 6).

Non-Folate Utilizing Enzymes Involved in Homocysteine Metabolism

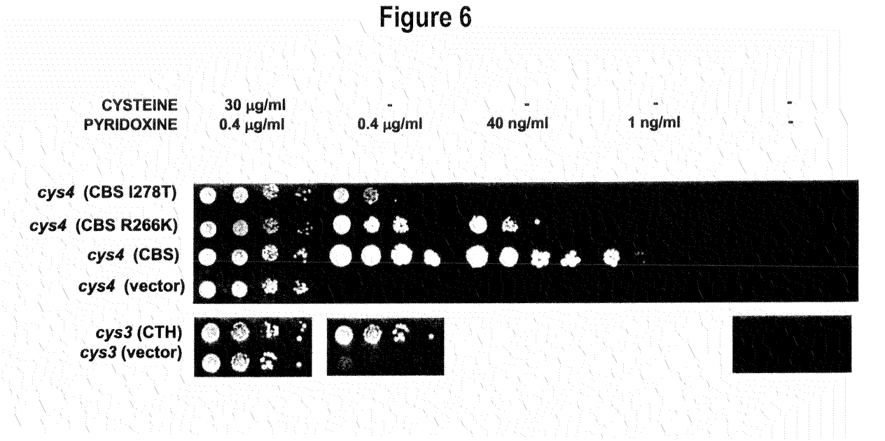

Cystathionine-β-Synthase (CBS) defects result in elevated homocysteine levels and Cystathionine-β-Lyase (CTH) SNPs have been similarly associated with elevated homocysteine (see, for example, Kraus et al., supra; Wang et al., 2004. Single nucleotide polymorphism in CTH associated with variation in plasma homocysteine concentration. Clin Genet 65:483-486). Although not folate-utilizing enzymes, both CBS and CTH depend on a vitamin B₆-cofactor, and impaired alleles pose a risk of dysfunctional folate/homocysteine metabolism. Impaired alleles of CBS and CTH are targets for B₆ therapy, analogous to folate therapy for MTHFR impaired alleles as described herein. Function and vitamin-responsiveness of CBS and CTH are recapitulated in the yeast complementation assay. (FIG. 6).

Vitamin B₆-Remediation of CBS Mutant Enzymes is Recapitulated in S. cerevisiae

Yeast strains were engineered to assay CTH and CBS as a function of intracellular vitamin B₆ (pyridoxine) concentration (FIG. 6). The S. cerevisiae orthologs for CTH and CBS are cys3 and cys4, respectively, whose defect results in cysteine auxotrophy. Enzymes were tested as a function of pyridoxine concentration in a manner similar to that described herein for MTHFR except that the strain background is defective for pyridoxine biosynthesis (sextuple-delete sno1Δ sno2Δ sno3Δ snz1Δ snz2Δ snz3Δ; Stolz et al., 2003. Tpn1p, the plasma membrane vitamin B₆ transporter of Saccharomyces cerevisiae. J Biol Chem 278:18990-18996) as well as either a cys3 or cys4 defect.

FIG. 6 shows qualitative yeast growth assays on solid media and demonstrates that both enzymes rescue the cognate yeast defect as a function of pyridoxine supplementation and that the vitamin-responsiveness of two homocystinuric alleles of CBS (1278T, R266K) is recapitulated in this complementation assay: these alleles become more sensitive than the wild-type enzyme to limiting B₆ levels and show correspondingly greater growth defects. The rescue of cysteine auxotrophy in the cys4 mutant by human CBS has been demonstrated previously (Kruger et al., 1995. A yeast assay for functional detection of mutations in the human cystathionine-β-synthase gene. Hum Mol Genet 4:1155-1161; Kruger et al., 1994. A yeast system for expression of human cystathionine beta-synthase: structural and functional conservation of the human and yeast genes. Proc Natl Acad Sci 91:6614-6618).

### Example 2

### Identification of Additional MTHFR Variants

DNA Sample Population. Genomic DNA was isolated from dried bloodspots (Guthrie Cards) of each of 250 newborns affected with a neural tube defect or each of 250 newborns not affected with a neural tube defect. The MTHFR exons in the isolated genomic DNA samples were sequenced as indicated in Example 1. Mutations that affect enzyme structure were identified from sequence data as mismatches against the consensus human genome sequence (NM_{—}005957). All substitutions are listed in Table A.

TABLE A
Additional MTHFR Variants

| GENE_position | Exon | Type | Function | Location | dB SNP id | Change |
|---|---|---|---|---|---|---|
| MTHFR_3921 | 2 | SNP | non-coding | 5′-UTR | rs34889587 | C/T |
| MTHFR_4059 | 2 | SNP | Synonymous | P39P | rs2066470 | C/T |
| MTHFR_4078 | 2 | SNP | Nonsynonymous | R46W | | C/T |
| MTHFR_4145 | 2 | SNP | Nonsynonymous | R68Q | rs2066472 | A/G |
| MTHFR_4181 | 2 | SNP | non-coding | IVS2 + 3 | rs1413355 | A/G |
| MTHFR_4234 | 2 | SNP | non-coding | IVS + 56 | | A/G |
| MTHFR_5699 | 3 | SNP | Synonymous | D92D | rs45546035 | C/T |
| MTHFR_5733 | 3 | SNP | Nonsynonymous | D104Y | | G/T |
| MTHFR_5840 | 3 | SNP | Synonymous | T139T | rs2066466 | A/G |
| MTHFR_5872 | 3 | SNP | Nonsynonymous | L150P | | C/T |
| MTHFR_6642 | 4 | SNP | non-coding | IVS3 − 95 | | C/T |
| MTHFR_6651 | 4 | SNP | non-coding | IVS3 − 86 | rs13306567 | C/G |
| MTHFR_6657 | 4 | SNP | non-coding | IVS3 − 80 | | C/T |
| MTHFR_6658 | 4 | SNP | non-coding | IVS3 − 79 | rs2066471 | A/G |
| MTHFR_6661 | 4 | SNP | non-coding | IVS3 − 76 | rs2066469 | A/G |
| MTHFR_6681 | 4 | indel | non-coding | IVS3 − 56 | | −/+ |
| | | | | | | deletion |
| | | | | | | AG |
| MTHFR_6774 | 4 | SNP | Synonymous | G171G | | A/C |
| MTHFR_10738 | 5 | SNP | Nonsynonymous | A222V | rs59514310 | C/T |
| MTHFR_10906 | 5 | SNP | non-coding | IVS5 + 53 | | C/T |
| MTHFR_11656 | 6 | SNP | non-coding | IVS5 − 55 | | C/T |
| MTHFR_11668 | 6 | SNP | non-coding | IVS5 − 43 | | C/T |
| MTHFR_11836 | 6 | SNP | Synonymous | A302A | rs13306555 | C/T |
| MTHFR_11902 | 6 | SNP | Synonymous | N324N | | C/T |
| MTHFR_12044 | 6 | SNP | non-coding | IVS6 + 83 | rs2066467 | A/G |
| MTHFR_12190 | 7 | SNP | non-coding | IVS6 − 6 | rs2066464 | A/G |
| MTHFR_12220 | 7 | SNP | Synonymous | S352S | rs2066462 | C/T |
| MTHFR_12232 | 7 | SNP | Synonymous | K356K | | A/G |
| MTHFR_12361 | 7 | SNP | non-coding | IVS7 + 31 | rs1994798 | C/T |
| MTHFR_12445 | 8 | SNP | non-coding | IVS7 − 76 | rs12121543 | G/T |
| MTHFR_12618 | 8 | SNP | Nonsynonymous | G422R | rs45571736 | A/G |
| MTHFR_12622 | 8 | indel | Frame Shift | E423fs | | −/+ |
| | | | | | | insertion G |
| MTHFR_12641 | 8 | SNP | Nonsynonymous | E429A | rs1801131 | A/C |
| MTHFR_12660 | 8 | SNP | Synonymous | F435F | rs57431061 | C/T |
| MTHFR_12759 | 8 | SNP | non-coding | IVS8 + 57 | | A/G |
| MTHFR_13040 | 9 | SNP | Nonsynonymous | R473W | | C/T |
| MTHFR_13099 | 9 | SNP | Synonymous | P492P | rs35653697 | A/G |
| MTHFR_13192 | 9 | SNP | non-coding | IVS9 + 39 | rs45515693 | C/T |
| MTHFR_14593 | 10 | SNP | non-coding | IV9 − 88 | | G/T |
| MTHFR_14601 | 10 | SNP | non-coding | IVS9 − 80 | rs17375901 | A/G |
| MTHFR_14612 | 10 | SNP | non-coding | IVS9 − 69 | | A/G |
| MTHFR_14705 | 10 | SNP | Nonsynonymous | R519C | rs45496998 | C/T |
| MTHFR_14814 | 10 | SNP | non-coding | IVS10 + 32 | rs45497396 | C/T |
| MTHFR_14817 | 10 | SNP | non-coding | IVS10 + 35 | rs58018465 | A/G |
| MTHFR_16114 | 12 | SNP | non-coding | IVS11 − 48 | rs56932901 | C/G |
| MTHFR_16136 | 12 | SNP | non-coding | IVS11 − 26 | rs45622739 | A/G |
| MTHFR_16170 | 12 | SNP | Synonymous | A587A | | C/T |
| MTHFR_16190 | 12 | SNP | Nonsynonymous | R594Q | rs58316272 | A/G |
| MTHFR_16367 | 12 | SNP | Nonsynonymous | T653M | rs35737219 | C/T |
| MTHFR_16368 | 12 | SNP | Synonymous | T653T | rs45572531 | A/G |
| MTHFR_16401 | 12 | SNP | non-coding | 3′UTR | | C/T |
| MTHFR_16451 | 12 | SNP | non-coding | 3′UTR | | C/T |

The functional impact of the MTHFR variants are tested using the in vivo yeast assay disclosed herein over a range of folate concentrations to observe functional effects as described in Example 1.

### Example 3

### Identification of ATIC, MTHFS, MAT1A, MAT2A and GART Variants

DNA Sample Population. Genomic DNA was isolated from dried bloodspots (Guthrie Cards) of each of 250 newborns affected with a neural tube defect or each of 250 newborns not affected with a neural tube defect. A total of 234 exons in 18 candidate genes from the folate/homocysteine metabolic pathway were sequenced. Sequencing and amplicon Mutations that affect enzyme structure were identified from sequence data as mismatches against the consensus human genome sequences listed in Table 2 for ATIC, MTHFS, MAT1A, MAT2A, and GART. All substitutions for ATIC, MTHFS, MAT1A, MAT2A, and GART are respectively listed in Tables B, C, D, E, and F.

TABLE B
ATIC Variants

| GENE_position | Exon | Type | Function | Location | dB SNP id | Change |
|---|---|---|---|---|---|---|
| 1089 | 1 | SNP | non-coding | 5′UTR | rs28366034 | C/T |
| 1100 | 1 | SNP | non-coding | 5′UTR | | C/T |
| 1114 | 1 | SNP | non-coding | 5′UTR | | C/T |
| 1116 | 1 | SNP | non-coding | 5′UTR | rs4535042 | T/C |
| 1133 | 1 | SNP | non-coding | 5′UTR | rs28366035 | C/G/T |
| | | | | | | (TRIALLELE) |
| 1152 | 1 | SNP | non-coding | 5′UTR | rs11550205 | C/T |
| 1160 | 1 | SNP | non-coding | 5′UTR | rs11550203 | C/T |
| 1179 | 1 | SNP | Nonsynonymous | A2V | | C/T |
| 1244 | 1 | indel | non-coding | IVS1 + 50 | | −/+ insertionC |
| 1270 | 1 | SNP | non-coding | IVS1 + 76 | | C/T |
| 1288 | 1 | SNP | non-coding | IVS1 + 94 | | G/A |
| 1301 | 1 | SNP | non-coding | IVS1 + 107 | | G/A |
| 1380 | 2 | SNP | non-coding | IVS1 − 151 | | A/G |
| 1396 | 2 | SNP | non-coding | IVS1 − 135 | | G/C |
| 1453 | 2 | SNP | non-coding | IVS1 − 78 | | C/T |
| 1506 | 2 | SNP | non-coding | IVS1 − 25 | | T/C |
| 1689 | 2 | SNP | non-coding | IVS2 + 32 | | T/A |
| 7227 | 3 | SNP | Nonsynonymous | G62R | | G/C |
| 7232 | 3 | indel | Nonsynonymous | G63fs | | −/+ insertionG |
| 7388 | 3 | SNP | non-coding | IVS3 + 121 | | T/A |
| 8756 | 4 | SNP | Nonsynonymous | N94S | | A/G |
| 8793 | 4 | SNP | non-coding | IVS4 + 28 | rs16853782 | A/G |
| 8808 | 4 | SNP | non-coding | IVS4 + 43 | | G/A |
| 14099 | 5 | SNP | non-coding | IVS4 − 176 | | C/T |
| 14136 | 5 | SNP | non-coding | IVS4 − 139 | rs3772077 | A/G |
| 14140 | 5 | SNP | non-coding | IVS4 − 135 | | C/A |
| 14144 | 5 | SNP | non-coding | IVS4 − 131 | | C/T |
| 14156 | 5 | SNP | non-coding | IVS4 − 119 | rs3772078 | A/G |
| 14183 | 5 | SNP | non-coding | IVS4 − 92 | | C/T |
| 14229 | 5 | SNP | non-coding | IVS4 − 46 | | A/G |
| 14238 | 5 | SNP | non-coding | IVS4 − 37 | | C/T |
| 14245 | 5 | SNP | non-coding | IVS4 − 30 | | A/C |
| 14260 | 5 | SNP | non-coding | IVS4 − 15 | | G/T |
| 14331 | 5 | SNP | Nonsynonymous | T116S | rs2372536 | G/C |
| 14489 | 5 | SNP | non-coding | IVS5 + 126 | | G/A |
| 14965 | 6 | SNP | non-coding | IVS5 − 56 | rs7563206 | C/T |
| 14970 | 6 | SNP | non-coding | IVS5 − 51 | | C/T |
| 15003 | 6 | SNP | non-coding | IVS5 − 18 | | G/A |
| 15040 | 6 | SNP | Synonymous | R133R | | A/G |
| 15043 | 6 | SNP | Synonymous | A134A | | T/C |
| 15149 | 6 | SNP | Nonsynonymous | T170A | | A/G |
| 15240 | 6 | SNP | non-coding | IVS6 + 68 | | A/G |
| 15826 | 7 | SNP | non-coding | IVS6 − 30 | rs6751557 | C/T |
| 15844 | 7 | SNP | non-coding | IVS6 − 12 | | C/T |
| 16063 | 7 | SNP | non-coding | IVS7 + 51 | | G/A |
| 21363 | 8 | SNP | non-coding | IVS7 − 53 | | A/G |
| 21372 | 8 | SNP | non-coding | IVS7 − 44 | | T/G |
| 21400 | 8 | SNP | non-coding | IVS7 − 16 | | A/G |
| 21521 | 8 | indel | Nonsynonymous | F265fs | | −/+ deletionT |
| 21611 | 8 | SNP | non-coding | IVS8 + 70 | | T/A |
| 22187 | 9 | SNP | non-coding | IVS8 − 197 | | G/A |
| 22273 | 9 | SNP | non-coding | IVS8 − 111 | | A/G |
| 22282 | 9 | indel | non-coding | IVS8 − 103 | | −/+ insertionA |
| 22283 | 9 | SNP | non-coding | IVS8 − 102 | rs12995526 | C/T |
| 22291 | 9 | SNP | non-coding | IVS8 − 94 | | G/A |
| 22342 | 9 | SNP | non-coding | IVS8 − 43 | | A/G |
| 22361 | 9 | SNP | non-coding | IVS8 − 24 | rs10179873 | A/G |
| 22512 | 9 | SNP | non-coding | IVS9 + 20 | | T/G |
| 22519 | 9 | SNP | non-coding | IVS9 + 27 | | G/T |
| 22538 | 9 | SNP | non-coding | IVS9 + 46 | | A/G |
| 22564 | 9 | indel | non-coding | IVS9 + 72 | | −/+ deletion |
| | | | | | | GGA |
| 22589 | 9 | SNP | non-coding | IVS9 + 97 | | G/T |
| 22686 | 9 | SNP | non-coding | IVS9 + 194 | rs10932606 | C/T |
| 22737 | 9 | SNP | non-coding | IVS9 + 245 | | A/G |
| 24992 | 11 | indel | non-coding | IVS10 − 79 | | −/+ insertionG |
| 25009 | 11 | SNP | non-coding | IVS10 − 62 | | A/G |
| 25220 | 11 | SNP | non-coding | IVS11 + 60 | rs13002576 | G/C |
| 27609 | 12 | SNP | non-coding | IVS11 − | rs16853823 | A/G |
| | | | | 206 | | |
| 27739 | 12 | SNP | non-coding | IVS11 − 76 | rs6721444 | C/A |
| 27757 | 12 | SNP | non-coding | IVS11 − 58 | | A/G |
| 27855 | 12 | SNP | Nonsynonymous | T380I | | C/T |
| 27985 | 12 | SNP | non-coding | IVS12 + 42 | | T/C |
| 28015 | 12 | SNP | non-coding | IVS12 + 72 | | A/G |
| 33785 | 13 | SNP | non-coding | IVS12 − 30 | rs13010249 | A/G |
| 33901 | 13 | SNP | Synonymous | N438N | | C/T |
| 33919 | 13 | SNP | non-coding | IVS13 + 12 | | G/A |
| 33920 | 13 | SNP | non-coding | IVS13 + 13 | | T/C |
| 33933 | 13 | SNP | non-coding | IVS13 + 26 | | C/T |
| 35723 | 14 | SNP | non-coding | IVS13 − 72 | | G/A |
| 35737 | 14 | SNP | non-coding | IVS13 − 58 | | C/A |
| 35742 | 14 | SNP | non-coding | IVS13 − 53 | | G/C |
| 35840 | 14 | SNP | Nonsynonymous | R456S | | C/A |
| 35885 | 14 | SNP | Nonsynonymous | P471S | rs56117859 | C/T |
| 35917 | 14 | SNP | Synonymous | G481G | | A/G |
| 35968 | 14 | SNP | Synonymous | T498T | | G/C |
| 35973 | 14 | SNP | Nonsynonymous | G500D | | G/A |
| 38338 | 15 | indel | non-coding | IVS15 + 53 | | −/+ deletion |
| | | | | | | GT |
| 38342 | 15 | SNP | non-coding | IVS15 + 57 | | C/G |
| 38437 | 16 | SNP | non-coding | IVS15 − | rs4672768 | G/A |
| | | | | 135 | | |
| 38582 | 16 | SNP | Nonsynonymous | A557V | | C/T |
| 38627 | 16 | SNP | Nonsynonymous | I572T | | T/C |
| 38667 | 16 | SNP | Synonymous | T585T | | G/A |
| 38725 | 16 | SNP | non-coding | 3′UTR | | T/C |

TABLE C
MTHFS Variants

| GENE_position | Exon | Type | Function | Location | dB SNP id | Change |
|---|---|---|---|---|---|---|
| MTHFS_8636 | 2 | SNP | Non-coding | IVS1 − 39 | rs16971502 | C/T |
| MTHFS_8808 | 2 | SNP | Nonsynonymous | R84Q | | A/G |
| MTHFS_8912 | 2 | SNP | Nonsynonymous | V119L | | C//G |
| MTHFS_8957 | 2 | SNP | Non-coding | IVS2 + 21 | | A/G |
| MTHFS_8998 | 2 | SNP | Non-coding | IVS2 + 62 | | A/G |
| MTHFS_52560 | 3 | SNP | Non-coding | IVS2 − 27 | | C/T |
| MTHFS_52811 | 3 | SNP | Nonsynonymous | T202A | rs8923 | A/G |
| | | | | H280D | | A/G |
| MTHFS_52878 | 3 | SNP | Non-coding | 3′UTR | | G/T |
| MTHFS_52902 | 3 | SNP | Non-coding | 3′UTR | | Change |

TABLE D
MAT1A Variants

| GENE_position | Exon | Type | Function | Location | dB SNP id | Change |
|---|---|---|---|---|---|---|
| MAT1A_5045 | 2 | SNP | non-coding | IVS1 − 45 | | A/T |
| MAT1A_5081 | 2 | SNP | non-coding | IVS1 − 9 | rs10887721 | C/G |
| MAT1A_5181 | 2 | SNP | non-coding | IVS2 + 14 | | A/G |
| MAT1A_5233 | 2 | SNP | non-coding | IVS2 + 66 | | A/G |
| MAT1A_6739 | 3 | SNP | Nonsynonymous | I90V | | A/G |
| MAT1A_6795 | 3 | SNP | non-coding | IVS3 + 32 | | G/T |
| MAT1A_9833 | 4 | SNP | non-coding | IVS3 − 54 | | C/T |
| MAT1A_10006 | 4 | SNP | non-coding | IVS4 + 7 | | C/T |
| MAT1A_10089 | 4 | SNP | non-coding | IVS4 + 90 | rs2282367 | C/T |
| MAT1A_10312 | 5 | SNP | non-coding | IVS4 − 51 | | C/T |
| MAT1A_10339 | 5 | SNP | non-coding | IVS4 − 24 | | A/G |
| MAT1A_10374 | 5 | SNP | Synonymous | F139F | | C/T |
| MAT1A_10383 | 5 | SNP | Synonymous | A142A | rs1143694 | C/T |
| MAT1A_10484 | 5 | SNP | Nonsynonymous | L176R | | G/T |
| MAT1A_10555 | 5 | SNP | non-coding | IVS5 + 49 | | A/C |
| MAT1A_14038 | 6 | SNP | non-coding | IVS5 − 47 | | A/G |
| MAT1A_14114 | 6 | SNP | Synonymous | G193G | | C/T |
| MAT1A_14177 | 6 | SNP | Synonymous | T214T | | A/G |
| MAT1A_15424 | 7 | SNP | non-coding | IVS6 − 56 | | A/C |
| MAT1A_15500 | 7 | SNP | Synonymous | G263G | | C/T |
| MAT1A_15581 | 7 | SNP | Synonymous | V290V | rs60582388 | A/G |
| MAT1A_15593 | 7 | SNP | Synonymous | A294A | rs59923268 | C/T |
| MAT1A_15596 | 7 | SNP | Synonymous | A295A | rs17851642 | A/T |
| MAT1A_15646 | 7 | SNP | Nonsynonymous | R312Q | | A/G |
| MAT1A_15706 | 7 | SNP | non-coding | IVS7 + 44 | | C/T |
| MAT1A_15715 | 7 | SNP | non-coding | IVS7 + 53 | | AG |
| MAT1A_15730 | 7 | indel | non-coding | IVS7 + 68 | | −/+ |
| | | | | | | deletionA |
| MAT1A_15758 | 7 | SNP | non-coding | IVS7 + 96 | | C/T |
| MAT1A_15760 | 7 | SNP | non-coding | IVS7 + 98 | rs10788545 | C/T |
| MAT1A_16133 | 8 | SNP | Synonymous | F353F | | C/T |
| MAT1A_16173 | 8 | SNP | non-coding | IVS8 + 14 | rs2994388 | C/T |
| MAT1A_16174 | 8 | SNP | non-coding | IVS8 + 15 | | A/G |
| MAT1A_16218 | 8 | SNP | non-coding | IVS8 + 59 | | A/T |
| MAT1A_16752 | 9 | SNP | non-coding | IVS8 − 44 | rs57820177 | C/T |
| MAT1A_16841 | 9 | SNP | Synonymous | Y377Y | rs57257983 | C/T |
| MAT1A_16965 | 9 | SNP | non-coding | 3′ UTR | rs7087728 | C/T |
| MAT1A_16971 | 9 | SNP | non-coding | 3′ UTR | | G/T |

TABLE E
MAT2A Variants

| GENE_position | Exon | Type | Function | Location | dB SNP id | Change |
|---|---|---|---|---|---|---|
| MAT2A_2871 | 2 | SNP | non-coding | IVS1 − 48 | | A/C |
| MAT2A_2873 | 2 | indel | non-coding | IVS1-50 | | −/+ |
| | | | | | | insertion |
| | | | | | | ATAC |
| MAT2A_2939 | 2 | SNP | Synonymous | Q36Q | | A/G |
| MAT2A_3047 | 3 | SNP | non-coding | IVS2 − 48 | rs58507836 | A/G |
| MAT2A_3287 | 3 | SNP | non-coding | IVS3 + 70 | | A/G |
| MAT2A_3394 | 4 | SNP | non-coding | IVS3 − 79 | | C/T |
| MAT2A_3466 | 4 | SNP | non-coding | IVS3 − 7 | | C/G |
| MAT2A_3498 | 4 | SNP | Synonymous | V106V | | G/T |
| MAT2A_3617 | 4 | SNP | non-coding | IVS4 + 32 | rs62620249 | C/T |
| MAT2A_3650 | 5 | SNP | non-coding | IVS4 − 19 | | A/G |
| MAT2A_3704 | 5 | SNP | Synonymous | E147E | | A/G |
| MAT2A_3963 | 6 | SNP | non-coding | IVS5 − 32 | rs1078005 | A/G |
| MAT2A_4174 | 6 | SNP | Synonymous | H243H | | C/T |
| MAT2A_4428 | 7 | SNP | Synonymous | R264R | rs1078004 | C/G |
| MAT2A_4449 | 7 | SNP | Synonymous | Y271Y | | C/T |
| MAT2A_4476 | 7 | SNP | Synonymous | G280G | | C/T |
| MAT2A_4608 | 7 | SNP | non-coding | IVS7 + 21 | | C/G |
| MAT2A_4660 | 8 | SNP | non-coding | IVS7 − 81 | | C/G |
| MAT2A_4692 | 8 | SNP | non-coding | IVS7 − 49 | | A/G |
| MAT2A_4931 | 8 | indel | non-coding | IVS8 + 53 | | −/+ |
| | | | | | | insertion |
| | | | | | | GT |
| MAT2A_5313 | 9 | SNP | non-coding | IVS8 − | | C/T |
| | | | | 199 | | |
| MAT2A_5460 | 9 | indel | non-coding | IVS8 − 54 | | −/+ |
| | | | | | | insertionT |
| MAT2A_5480 | 9 | SNP | non-coding | IVS8 − 33 | | C/T |

TABLE F
GART Variants

| GENE_position | Exon | Type | Function | Location | dB SNP id | Change |
|---|---|---|---|---|---|---|
| GART_3782 | 2 | SNP | non-coding | 5′UTR | | G/T |
| GART_3842 | 2 | SNP | Nonsynonymous | T16M | | C/T |
| GART_7745 | 3 | SNP | non-coding | IVS2 − 46 | | G/T |
| GART_7984 | 3 | SNP | non-coding | IVS3 + 98 | | C/T |
| GART_10720 | 5 | SNP | Nonsynonymous | A161G | rs35035222 | C/G |
| GART_10775 | 5 | SNP | non-coding | IVS5 + 9 | | A/G |
| GART_11521 | 6 | SNP | non-coding | IVS5 − 33 | | A/T |
| GART_11522 | 6 | SNP | non-coding | IVS5 − 32 | | A/T |
| GART_11541 | 6 | SNP | non-coding | IVS5 − 13 | | A/C |
| GART_12356 | 7 | SNP | non-coding | IVS7 + 4 | | C/T |
| GART_14200 | 8 | SNP | Synonymous | I250I | | C/T |
| GART_14273 | 8 | SNP | non-coding | IVS8 + 12 | | C/T |
| GART_14282 | 8 | SNP | non-coding | IVS8 + 21 | | A/G |
| GART_14739 | 10 | SNP | non-coding | IVS9 − 37 | | A/C |
| GART_14781 | 10 | SNP | Synonymous | I301I | | C/T |
| GART_18055 | 11 | SNP | non-coding | IVS10 − 55 | | C/T |
| GART_18064 | 11 | SNP | non-coding | IVS10 − 46 | | A/G |
| GART_18130 | 11 | SNP | Nonsynonymous | L363I | | A/C |
| GART_18142 | 11 | SNP | Nonsynonymous | V367M | | A/G |
| GART_18197 | 11 | SNP | Nonsynonymous | R385K | | A/G |
| GART_18232 | 11 | SNP | Nonsynonymous | I397V | | A/G |
| GART_18304 | 11 | SNP | Nonsynonymous | V421I | rs60421747 | A/G |
| GART_18401 | 11 | SNP | non-coding | IVS11 + 60 | | A/T |
| GART_20794 | 12 | SNP | non-coding | IVS11 − 34 | rs2834234 | A/G |
| GART_20812 | 12 | SNP | non-coding | IVS11 − 16 | | A/G |
| GART_20825 | 12 | SNP | non-coding | IVS11 − 3 | | C/T |
| GART_20862 | 12 | SNP | Nonsynonymous | A445T | | A/G |
| GART_22073 | 13 | SNP | non-coding | IVS12 − 22 | rs2834232 | C/T |
| GART_22481 | 14 | SNP | non-coding | IVS13 − 67 | | A/G |
| GART_22521 | 14 | SNP | non-coding | IVS13 − 27 | rs2834232 | A/G |
| GART_22573 | 14 | SNP | Nonsynonymous | D510G | rs35927582 | A/G |
| GART_25425 | 15 | SNP | non-coding | IVS14 − 77 | | A/G |
| GART_25433 | 15 | SNP | non-coding | IVS14 − 69 | | C/G |
| GART_25601 | 15 | SNP | Nonsynonymous | H601R | | A/G |
| GART_25694 | 15 | SNP | Nonsynonymous | A632V | rs59920090 | C/T |
| GART_25720 | 15 | SNP | Nonsynonymous | P641A | rs34588874 | C/G |
| GART_25867 | 16 | SNP | non-coding | IVS15 − | | C/T |
| | | | | 102 | | |
| GART_25912 | 16 | SNP | non-coding | IVS15 − 57 | | C/T |
| GART_25951 | 16 | SNP | non-coding | IVS15 − 18 | | C/T |
| GART_25956 | 16 | indel | non-coding | IVS15 − 13 | | −/+ |
| | | | | | | deletion |
| | | | | | | CT |
| GART_26127 | 16 | SNP | non-coding | IVS16 + 6 | | A/G |
| GART_26195 | 16 | SNP | non-coding | IVS16 + 74 | | C/G |
| GART_31619 | 17 | SNP | non-coding | IVS16 − 33 | rs7281488 | A/G |
| GART_31627 | 17 | SNP | non-coding | IVS16 − 25 | | A/T |
| GART_31641 | 17 | SNP | non-coding | IVS16 − 11 | | A/G |
| GART_31799 | 17 | SNP | Nonsynonymous | D752G | rs8971 | A/G |
| GART_31887 | 17 | SNP | non-coding | IVS17 + 29 | | C/T |
| GART_31902 | 17 | SNP | non-coding | IVS17 + 44 | | A/G |
| GART_31933 | 17 | SNP | non-coding | IVS17 + 75 | | A/C |
| GART_33173 | 18 | SNP | non-coding | IVS17 − 17 | | A/G |
| GART_33264 | 18 | SNP | Nonsynonymous | L797M | | A/C |
| GART_33286 | 18 | SNP | Nonsynonymous | E804A | | A/C |
| GART_36963 | 19 | SNP | non-coding | IVS18 − 43 | | A/G |
| GART_36964 | 19 | SNP | non-coding | IVS18 − 42 | | A/T |
| GART_36967 | 19 | SNP | non-coding | IVS18 − 39 | rs2070390 | A/T |
| GART_37428 | 20 | SNP | Synonymous | Y868Y | | C/T |
| GART_37433 | 20 | SNP | Nonsynonymous | N870S | | A/G |
| GART_38709 | 21 | SNP | non-coding | IVS21 + 11 | rs2070388 | C/G |
| GART_38762 | 22 | SNP | non-coding | IVS21 − 33 | | A/G |
| GART_38914 | 22 | SNP | Synonymous | A987A | | A/C |
| GART_38989 | 22 | SNP | non-coding | 3′ UTR | | C/G |

The functional impact of the ATIC, MTHFS, MAT1A, MAT2A, and GART variants are tested over a range of folate concentrations using the disclosed in vivo yeast assay to observe functional effects as described in Example 1 and using the appropriate yeast strain backgrounds as described in Table 1.

All citations are expressly incorporated herein in their entirety by reference.

TABLE 3
Spectrum of nonsynonymous MTHFR alleles observed from
sampling over 500 unselected individuals of diverse ethnicity

| | Length (bp) | | | | |
|---|---|---|---|---|---|
| Exon | Reference | Alleles Sequenced | Variant (codon) | | Occurrences* |
| 1 | 236** | 1070 | None | | |
| 2 | 239 | 1016 | M110I (atg→atc) | 1 | novel |
| | | | R134C (cgc→tgc) | 1 | 25 |
| 3 | 111 | 1068 | None | | |
| 4 | 194 | 1050 | A222V (gcc→gtc) | 308 | 26 |
| | | | H213R (cac→cgc) | 1 | novel |
| | | | D223N (gat→aat) | 1 | novel |
| 5 | 251 | 1056 | D291N (gat→aat) | 1 | novel |
| 6 | 135 | 1042 | None | | |
| 7 | 181 | 1062 | E429A (gaa→gca) | 251 | 27 |
| | | | G422R (ggg→agg) | 3 | 28 |
| 8 | 183 | 1058 | None | | |
| 9 | 102 | 1072 | R519C (cgc→tgc) | 2 | novel |
| | | | R519L (cgc→ctc) | 2 | novel |
| 10 | 120 | 1072 | M581I (atg→ata) | 1 | 29 |
| 11 | 219** | 1076 | R594Q (cgg→cag) | 47 | 30 |
| | | | T653M (acg→atg) | 4 | 31 |
| | | | Q648P (cag→ccg) | 1 | novel |

**for exons 1 and 11, only the length of the coding portion of the exon is given

We claim:

1. An in vivo method of screening for an impaired allele of an enzyme-encoding gene remediable by cofactor administration, comprising:
i) introducing into a yeast cell a test allele of the enzyme-encoding gene, wherein the yeast cell comprises a first mutation in a first gene that is functionally homologous to the enzyme-encoding gene, and a second mutation in a second gene or group of genes that renders the yeast cell dependent upon supplementation with a cofactor required for enzyme function, wherein the first mutation alters a measurable characteristic of the yeast related to the function of the first gene;
ii) supplementing the growth medium with the cofactor; and
iii) detecting less restoration of the measurable characteristic in the presence of the test allele than in the presence of the wildtype enzyme, thereby detecting incomplete complementation of the first gene mutation by the test allele and identifying the test allele as an impaired allele.

2. The method according to claim 1, further comprising titrating the amount of supplemented cofactor to determine if the test allele is cofactor sensitive.

3. The method according to claim 1, wherein the yeast is diploid.

4. The method according to claim 1, wherein the diploid yeast is heterozygous for the test allele of an enzyme-encoding gene.

5. The method according to claim 1, wherein the first gene is met13, the second gene is fol3, the cofactor is folate, the measurable characteristic is growth, and the enzyme-encoding gene is selected from the group consisting of MTHFR, MAT1A, MAT2A, GART, MTHFS and ATIC.

6. The method according to claim 1, wherein the first gene is cys3, the second group of genes is sextuple-delete sno1Δ sno2Δ sno3Δ snzlA snz2Δ snz3Δ, the enzyme encoding gene is CTH, the cofactor is vitamin B6, and the measurable characteristic is growth.

7. The method according to claim 1, wherein the first gene is cys4, the second group of genes is sextuple-delete sno1Δ sno2Δ sno3Δ snz1Δ snz2Δ snz3Δ, the enzyme encoding gene is CBS, the cofactor is vitamin B6, and the measurable characteristic is growth.

8. A method of detecting a predisposition to a cofactor-dependent enzyme deficiency, comprising:
i) obtaining a sample from said subject;
ii) detecting the presence or absence of a plurality of cofactor remediable impaired alleles of at least one enzyme-encoding gene;
wherein the presence of at least one impaired allele indicates that the subject is at risk of a cofactor-dependent enzyme deficiency.

9. A method for identifying and/or characterizing an enzyme deficiency within a metabolic pathway in a subject, comprising
i) obtaining a sample from said subject;
ii) detecting the presence or absence of a plurality of impaired alleles of at least one enzyme-encoding gene in said pathway;
wherein the presence of an impaired allele indicates that the subject has a remediable enzyme deficiency.

10. The method according to claim 8 or 9, wherein said impaired alleles are low-frequency alleles.

11. The method according to claim 8 or 9, wherein said impaired alleles are from multiple enzyme-encoding genes in said pathway.

12. The method according to claim 8 or 9, wherein said plurality of impaired alleles are identified by a method according to any one of claims 1 to 7.

13. A method for treating a metabolic enzyme deficiency in a subject, comprising:
i) obtaining a sample from said subject;
ii) detecting the presence or absence of a plurality of impaired alleles of at least one enzyme-encoding gene; and
iii) administering a cofactor supplement to said subject based the presence of at least one impaired allele.

14. The method according to any one of claims 8-13, wherein the metabolic pathway is homocysteine, the vitamin is folate, and the impaired alleles are selected from the group consisting of M110I, H213R, D223N, D291N, R519C, R519L, and Q648P in human MTHFR.

15. The method according to any one of claims 8-13, wherein the metabolic pathway is homocysteine, the vitamin is folate, and the impaired alleles are selected from the group consisting of R84Q, V 119L and T202A in human MTHFS.

16. The method according to any one of claims 8-13, wherein the metabolic pathway is homocysteine, the vitamin is folate, and the impaired alleles are selected from the group consisting of 190V, L176R and R312Q in human MAT1A.

17. The method according to any one of claims 8-13, wherein the metabolic pathway is homocysteine, the vitamin is folate, and the impaired alleles are selected from the group consisting of T16M, A161G, L3631, V367M, R385K, 1397V, V4211, A445T, D510G, H601R, A632V, P641A, D752G, L797M, E804A, and N870S in human GART.

18. A kit for evaluating remediable enzyme deficiencies in a metabolic pathway, comprising a plurality of nucleic acid probes for detecting low-frequency remediable impaired alleles in enzyme-encoding genes in said metabolic pathway.

19. The kit according to claim 18, wherein said impaired alleles are identified by the method according to any one of claims 1 to 7.

20. An isolated nucleic acid comprising an impaired allele mutation or the complement thereof, wherein said impaired allele mutation is selected from the group consisting of nucleotide 4078 of the MTHFR gene; nucleotide 4234 of the MTHFR gene; nucleotide 5733 of the MTHFR gene; nucleotide 5872 of the MTHFR gene; nucleotide 6642 of the MTHFR gene; nucleotide 6657 of the MTHFR gene; nucleotide 6681 of the MTHFR gene; nucleotide 6774 of the MTHFR gene; nucleotide 10906 of the MTHFR gene; nucleotide 11656 of the MTHFR gene; nucleotide 11668 of the MTHFR gene; nucleotide 11902 of the MTHFR gene; nucleotide 12232 of the MTHFR gene; nucleotide 2622 of the MTHFR gene; nucleotide 12759 of the MTHFR gene; nucleotide 13040 of the MTHFR gene; nucleotide 14593 of the MTHFR gene; nucleotide 14612 of the MTHFR gene; nucleotide 14705 of the MTHFR gene; nucleotide 13170 of the MTHFR gene; nucleotide 116401 of the MTHFR gene; wherein the sequence of the SNP is provided in Table A.

21. An isolated nucleic acid comprising an impaired allele mutation or the complement thereof, wherein said impaired allele mutation is selected from the group consisting of nucleotide 1100 of the ATIC gene; nucleotide 1114 of the ATIC gene; nucleotide 1179 of the ATIC gene; nucleotide 1244 of the ATIC gene; nucleotide 1270 of the ATIC gene; nucleotide 1288 of the ATIC gene; nucleotide 1301 of the ATIC gene; nucleotide 1380 of the ATIC gene; nucleotide 1396 of the ATIC gene; nucleotide 1453 of the ATIC gene; nucleotide 1506 of the ATIC gene; nucleotide 1689 of the ATIC gene; nucleotide 7227 of the ATIC gene; nucleotide 7232 of the ATIC gene; nucleotide 7388 of the ATIC gene; nucleotide 8756 of the ATIC gene; nucleotide 8808 of the ATIC gene; nucleotide 14099 of the ATIC gene; nucleotide 14140 of the ATIC gene; nucleotide 14144 of the ATIC gene; nucleotide 14183 of the ATIC gene; nucleotide 14229 of the ATIC gene; nucleotide 14238 of the ATIC gene; nucleotide 14245 of the ATIC gene; nucleotide 14260 of the ATIC gene; nucleotide 14489 of the ATIC gene; nucleotide 14970 of the ATIC gene; nucleotide 15003 of the ATIC gene; nucleotide 15040 of the ATIC gene; nucleotide 15043 of the ATIC gene; nucleotide 15149 of the ATIC gene; nucleotide 15240 of the ATIC gene; nucleotide 15844 of the ATIC gene; nucleotide 16063 of the ATIC gene; nucleotide 21363 of the ATIC gene; nucleotide 21372 of the ATIC gene; nucleotide 21400 of the ATIC gene; nucleotide 21521 of the ATIC gene; nucleotide 21611 of the ATIC gene; nucleotide 22187 of the ATIC gene; nucleotide 22273 of the ATIC gene; nucleotide 22282 of the ATIC gene; nucleotide 22291 of the ATIC gene; nucleotide 22342 of the ATIC gene; nucleotide 22512 of the ATIC gene; nucleotide 22519 of the ATIC gene; nucleotide 22538 of the ATIC gene; nucleotide 22564 of the ATIC gene; nucleotide 22589 of the ATIC gene; nucleotide 22737 of the ATIC gene; nucleotide 24992 of the ATIC gene; nucleotide 25009 of the ATIC gene; nucleotide 27757 of the ATIC gene; nucleotide 27855 of the ATIC gene; nucleotide 27985 of the ATIC gene; nucleotide 28015 of the ATIC gene; nucleotide 33901 of the ATIC gene; nucleotide 33919 of the ATIC gene; nucleotide 33920 of the ATIC gene; nucleotide 33933 of the ATIC gene; nucleotide 35723 of the ATIC gene; nucleotide 35737 of the ATIC gene; nucleotide 35742 of the ATIC gene; nucleotide 35840 of the ATIC gene; nucleotide 35917 of the ATIC gene; nucleotide 35968 of the ATIC gene; nucleotide 35973 of the ATIC gene; nucleotide 38338 of the ATIC gene; nucleotide 38342 of the ATIC gene; nucleotide 38437 of the ATIC gene; nucleotide 38342 of the ATIC gene; nucleotide 38582 of the ATIC gene; nucleotide 38627 of the ATIC gene; nucleotide 38667 of the ATIC gene; and nucleotide 38725 of the ATIC gene; wherein the sequence of the nucleotide is provided in Table B.

22. An isolated nucleic acid comprising an impaired allele mutation or the complement thereof, wherein said impaired allele mutation is selected from the group consisting of nucleotide 8808 of the MTHFS gene; nucleotide 8912 of the MTHFS gene; nucleotide 8957 of the MTHFS gene; nucleotide 8998 of the MTHFS gene; nucleotide 52560 of the MTHFS gene; nucleotide 52878 of the MTHFS gene; and nucleotide 52902 of the MTHFS gene; wherein the sequence of the SNP is provided in Table C.

23. An isolated nucleic acid comprising an impaired allele mutation or the complement thereof, wherein said impaired allele mutation is selected from the group consisting of nucleotide 5045 of the MAT1A gene; nucleotide 5181 of the MAT1A gene; nucleotide 5233 of the MAT1A gene; nucleotide 6739 of the MAT1A gene; nucleotide 6795 of the MAT1A gene; nucleotide 9833 of the MAT1A gene; nucleotide 10006 of the MAT1A gene; nucleotide 10312 of the MAT1A gene; nucleotide 10339 of the MAT1A gene; nucleotide 10374 of the MAT1A gene; nucleotide 10484 of the MAT1A gene; nucleotide 10555 of the MAT1A gene; nucleotide 14038 of the MAT1A gene; nucleotide 14114 of the MAT1A gene; nucleotide 14177 of the MAT1A gene; nucleotide 15424 of the MAT1A gene; nucleotide 15500 of the MAT1A gene; nucleotide 15646 of the MAT1A gene; nucleotide 15706 of the MAT1A gene; nucleotide 15715 of the MAT1A gene; nucleotide 15730 of the MAT1A gene; nucleotide 15758 of the MAT1A gene; nucleotide 16133 of the MAT1A gene; nucleotide 16174 of the MAT1A gene; nucleotide 15706 of the MAT1A gene; nucleotide 15715 of the MAT1A gene; nucleotide 15730 of the MAT1A gene; nucleotide 15758 of the MAT1A gene; nucleotide 16133 of the MAT1A gene; nucleotide 16174 of the MAT1A gene; nucleotide 16218 of the MAT1A gene; wherein the sequence of the SNP is provided in Table D.

24. An isolated nucleic acid comprising an impaired allele mutation or the complement thereof, wherein said impaired allele mutation is selected from the group consisting of nucleotide 2871 of the MAT2A gene; nucleotide 2873 of the MAT2A gene; nucleotide 2939 of the MAT2A gene; nucleotide 3287 of the MAT2A gene; nucleotide 3394 of the MAT2A gene; nucleotide 3466 of the MAT2A gene; nucleotide 3498 of the MAT2A gene; nucleotide 3650 of the MAT2A gene; nucleotide 3704 of the MAT2A gene; nucleotide 4174 of the MAT2A gene; nucleotide 4449 of the MAT2A gene; nucleotide 4476 of the MAT2A gene; nucleotide 4608 of the MAT2A gene; nucleotide 4660 of the MAT2A gene; nucleotide 4692 of the MAT2A gene; nucleotide 4931 of the MAT2A gene; nucleotide 5313 of the MAT2A gene; nucleotide 5460 of the MAT2A gene; and nucleotide 5480 of the MAT2A gene; wherein the sequence of the SNP is provided in Table E.

25. An isolated nucleic acid comprising an impaired allele mutation or the complement thereof, wherein said impaired allele mutation is selected from the group consisting of nucleotide 3782 of the GART gene; nucleotide 3842 of the GART gene; nucleotide 7745 of the GART gene; nucleotide 7984 of the GART gene; nucleotide 10775 of the GART gene; nucleotide 11521 of the GART gene; nucleotide 11522 of the GART gene; nucleotide 11541 of the GART gene; nucleotide 12356 of the GART gene; nucleotide 14200 of the GART gene; nucleotide 14273 of the GART gene; nucleotide 14282 of the GART gene; nucleotide 14739 of the GART gene; nucleotide 14781 of the GART gene; nucleotide 18055 of the GART gene; nucleotide 18064 of the GART gene; nucleotide 18130 of the GART gene; nucleotide 18142 of the GART gene; nucleotide 18197 of the GART gene; nucleotide 18232 of the GART gene; nucleotide 18401 of the GART gene; nucleotide 20812 of the GART gene; nucleotide 20825 of the GART gene; nucleotide 16174 of the GART gene; nucleotide 15706 of the GART gene; nucleotide 20862 of the GART gene; nucleotide 22481 of the GART gene; nucleotide 22521 of the GART gene; nucleotide 25425 of the GART gene; nucleotide 25433 of the GART gene; nucleotide 25601 of the GART gene; nucleotide 25867 of the GART gene; nucleotide 25912 of the GART gene; nucleotide 25951 of the GART gene; nucleotide 25956 of the GART gene; nucleotide 26127 of the GART gene; nucleotide 26195 of the GART gene; nucleotide 31627 of the GART gene; nucleotide 31641 of the GART gene; nucleotide 31887 of the GART gene; nucleotide 31902 of the GART gene; nucleotide 31933 of the GART gene; nucleotide 33173 of the GART gene; nucleotide 33264 of the GART gene; nucleotide 31933 of the GART gene; nucleotide 33173 of the GART gene; nucleotide 33264 of the GART gene; nucleotide 33286 of the GART gene; nucleotide 36963 of the GART gene; nucleotide 36964 of the GART gene; nucleotide 37428 of the GART gene; nucleotide 37433 of the GART gene; nucleotide 38762 of the GART gene; nucleotide 38914 of the GART gene; and nucleotide 38989 of the GART gene; wherein the sequence of the SNP is provided in Table F.

26. An isolated nucleic acid comprising an impaired allele mutation or the complement thereof, wherein said impaired allele mutation is selected from the group consisting of M110I, H213R, D223N, D291N, R519C, R519L, and Q648P.

27. A method of screening for risk of a condition or disease associated with aberrant folate/homocysteine metabolism, comprising detecting an impaired allele using the method of claims 8-13.

28. The method according to claim 27, wherein the disease or condition is selected from the group consisting of cardiovascular disease, coronary artery disease, ischemic stroke, atherosclerosis, neural tube defects, orofacial clefts, pre-eclampsia, pre-term delivery/low birthweight, recurrent early spontaneous abortion, thrombosis, retinal artery occlusion, down's syndrome, colorectal cancer, breast cancer, lung cancer, prostate cancer, depression, schizophrenia, Alzheimer's disease/dementia, age-related macular degeneration, and glaucoma.

28. A method of screening for chemotherapeutic response potential, comprising detecting an impaired allele of a gene selected from the group consisting of MTHFR and GART.

29. A method of screening for chemotherapeutic toxicity, comprising detecting an impaired allele of gene selected from the group consisting of MTHFR and GART.

30. An array for detecting an impaired allele of a gene in the folate/homocysteine metabolic pathway, comprising an isolated nucleic acid according to any one of claims 20-25.
